(19) United States
(12) Patent Application Publication
(10) Pub. No.: US 2021/0070875 A1
(43) Pub. Date: Mar. 11, 2021

(54) ANTI-C5AR ANTIBODY AND PREPARATION METHOD AND USE THEREOF

(71) Applicant: SHANGHAI PHARMAEXPLORER CO., LTD., Shanghai (CN)

(72) Inventors: Cuiqing YANG, Shanghai (CN); Qing DUAN, Shanghai (CN); Lina XU, Shanghai (CN); Hongzhuan GU, Shanghai (CN); Lile LIU, Shanghai (CN)

(21) Appl. No.: 16/965,209

(22) PCT Filed: Jan. 10, 2017
(86) PCT No.: PCT/CN2017/070713

§ 371 Date: Jul. 27, 2020

(30) Foreign Application Priority Data
Jan. 12, 2016 (CN) 201610018727.1

(51) Int. Cl. C07K 16/28; G01N 33/68

(52) U.S. Cl. CPC C07K 16/2896; C07K 2317/92; C07K 2317/565; G01N 33/6893

(57) ABSTRACT

The present invention provides an anti-C5aR antibody and a preparation method and use thereof. The anti-C5aR antibody comprises one or more of the followings: a heavy chain CDR1, heavy chain CDR2 or heavy chain CDR3 of a heavy chain variable region of an anti-C5aR antibody, and one or more of the followings: a light chain CDR1, light chain CDR2 or light chain CDR3 of a light chain variable region of the anti-C5aR antibody. The anti-C5aR antibody has a high affinity to human C5aR protein and can block the binding of the C5aR protein to a C5a, thus downregulating or switching off a corresponding signaling pathway, stopping C5a directed migration of a neutrophil in vitro and inhibiting chemotaxis of the neutrophil.

The present application claims the priority for Chinese patent application CN 201610018727.1 filed on Jan. 12, 2016. The aforementioned Chinese patent application is incorporated into the present application by reference in its entirety.

### TECHNICAL FIELD

The present invention relates to the field of antibodies, in particular to an anti-C5aR antibody and a preparation method and the use thereof.

### BACKGROUND ARTS

The complement system is a protein reaction system consisting of more than 30 plasma proteins and membrane proteins, which is widely exist in the blood and tissue fluid and on the cell surface, and has a precise regulatory mechanism. The main physiological function of the complement system is promoting the phagocytic ability of a phagocyte and lysing a target cell, thus the complement system is an important component of the body's immune defense mechanism.

The ligand C5a of C5aR is an activated product of the complement system, and is an important medium and chemokine of an inflammatory reaction. The binding of C5aR and the ligand C5a thereof induces an inflammatory reaction, influences the coagulation and fibrinolysis systems, and causes the damage of normal tissue cells, thereby involving the pathological process of various diseases. C5a is a medium showing the strongest anaphylatoxin effect among the complement cleavage fragments, and the anaphylatoxin effect of which is 20 folds and 2500 folds of that of C3a and C4a respectively. In addition, C5a also can increase the vasopermeability, and stimulate the smooth muscle to contract. The high concentration of C5a is a chemotactic agent for a neutrophil, eosinophil and monocyte, and can induce the cells to migrate in the direction of the concentration gradient. C5a has an obvious enhancement effect on an immune response, and can induce a monocyte to secrete interleukins IL-1, IL-6, and IL-8 and the expression of cytokines such as a tumor necrosis factor α (TNF-α), promote the T cell proliferation induced by an antigen and an allogeneic antigen, and promote B cells to produce an antibody.

C5aR is a member of seven-transmembrane G-protein coupled receptor family. C5aR is a receptor having a high affinity for C5a with Kd being about 1 nM, and it is located on different types of cells, including leukocytes. The amount of receptors of each cell is very high, and there are up to 200,000 sites on each leukocyte. C5a further has another receptor, referred to as C5a like receptor (C5L2), but since it does not contain G proteins, it is also called as GPR77. GPR77 is a member of C5a receptor subfamily, which has 35% amino acid sequence homology with CD88, but currently the biological function thereof is still ambiguous.

C5aR involves the pathogenesis of various diseases including rheumatic arthritis, psoriasis, sepsis, reperfusion injury and adult respiratory distress syndrome (Gerard and Gerard, 1994; Murdoch and Finn, 2000). How to block the transduction of C5aR signal to the downstream signaling pathway, thereby reducing the inflammatory reaction is always a hot issue of immunological research. Currently, antagonists of C5aR and C5a are mainly classified as anti-C5a antibody, anti-C5aR antibody, small molecule antagonist, C5a anti-sense peptide, C5a mutant or bacteria-derived chemotaxis inhibitory protein, etc. Currently, there are still no novel drugs which are related to the antagonist against C5aR and a ligand thereof in the market.

### Content of the Present Invention

The technical problem to be solved in the present invention is for overcoming the existing deficiency of lacking an anti-C5aR antibody, thus providing an anti-C5aR antibody with a high affinity and strong specificity and a preparation method and the use thereof. The anti-C5aR antibody has a high affinity to human C5aR protein, can effectively inhibit or block the binding of C5aR to C5a, and significantly reduce a calcium flow signal, thus downregulating or switching off a corresponding signaling pathway, stopping C5a directed migration of a neutrophil in vitro and inhibiting chemotaxis of the neutrophil, thereby bringing hope to the treatment of inflammation, vascular or nervous system diseases.

The inventors uses human C5aR protein as an immunogen, and uses optimized hybridoma techniques to clone heavy chain variable region genes and light chain variable region genes of the antibody, wherein the variable region genes can be grafted to the constant region genes of the human antibody to form a human and mouse chimeric antibody, i.e. a lead antibody of the anti-C5aR antibody. Then a series of initial production, purification, detection and identification of the lead antibody are performed, and the anti-C5aR antibody is obtained. The anti-C5aR antibody has a high affinity to proteins such as human C5aR protein, can effectively inhibit or block the binding of C5aR to C5a and significantly reduce a calcium flow signal, and can significantly inhibit chemotaxis of a neutrophil. Then the amino acid sequences of the heavy chain variable region and light chain variable region of the anti-C5aR antibody can be obtained by sequencing using molecular biology methods.

The present invention provides an isolated protein comprising one or more of the followings: a heavy chain CDR1, heavy chain CDR2 or heavy chain CDR3 of an anti-C5aR antibody, and/or, one or more of the followings: a light chain CDR1, light chain CDR2 or light chain CDR3 of the anti-C5aR antibody, wherein the amino acid sequence of the heavy chain CDR1 is as shown in SEQ ID No. 2, SEQ ID No. 10, SEQ ID No. 18, SEQ ID No. 26, SEQ ID No. 34, SEQ ID No. 42 or SEQ ID No. 50 in the sequence listing; the amino acid sequence of the heavy chain CDR2 is as shown in SEQ ID No. 3, SEQ ID No. 11, SEQ ID No. 19, SEQ ID No. 27, SEQ ID No. 35, SEQ ID No. 43 or SEQ ID No. 51 in the sequence listing; the amino acid sequence of the heavy chain CDR3 is as shown in SEQ ID No. 4, SEQ ID No. 12, SEQ ID No. 20, SEQ ID No. 28, SEQ ID No. 36, SEQ ID No. 44 or SEQ ID No. 52 in the sequence listing; the amino acid sequence of the light chain CDR1 is as shown in SEQ ID No. 6, SEQ ID No. 14, SEQ ID No. 22, SEQ ID No. 30, SEQ ID No. 38, SEQ ID No. 46 or SEQ ID No. 54 in the sequence listing; the amino acid sequence of the light chain CDR2 is as shown in SEQ ID No. 7, SEQ ID No. 15, SEQ ID No. 23, SEQ ID No. 31, SEQ ID No. 39, SEQ ID No. 47 or SEQ ID No. 55 in the sequence listing; the amino acid sequence of the light chain CDR3 is as shown in SEQ ID No. 8, SEQ ID No. 16, SEQ ID No. 24, SEQ ID No. 32, SEQ ID No. 40, SEQ ID No. 48 or SEQ ID No. 56 in the sequence listing;

or the amino acid sequence of the heavy chain CDR1 is as shown in an amino acid sequence which has at least 80% sequence homology with the amino acid sequence shown in SEQ ID No. 2, SEQ ID No. 10, SEQ ID No. 18, SEQ ID No. 26, SEQ ID No. 34, SEQ ID No. 42 or SEQ ID No. 50 in the sequence listing; the amino acid sequence of the heavy chain CDR2 is as shown in an amino acid sequence which has at least 80% sequence homology with the amino acid sequence shown in SEQ ID No. 3, SEQ ID No. 11, SEQ ID No. 19, SEQ ID No. 27, SEQ ID No. 35, SEQ ID No. 43 or SEQ ID No. 51 in the sequence listing; the amino acid sequence of the heavy chain CDR3 is as shown in an amino acid sequence which has at least 80% sequence homology with the amino acid sequence shown in SEQ ID No. 4, SEQ ID No. 12, SEQ ID No. 20, SEQ ID No. 28, SEQ ID No. 36, SEQ ID No. 44 or SEQ ID No. 52 in the sequence listing; the amino acid sequence of the light chain CDR1 is as shown in an amino acid sequence which has at least 80% sequence homology with the amino acid sequence shown in SEQ ID No. 6, SEQ ID No. 14, SEQ ID No. 22, SEQ ID No. 30, SEQ ID No. 38, SEQ ID No. 46 or SEQ ID No. 54 in the sequence listing; the amino acid sequence of the light chain CDR2 is as shown in an amino acid sequence which has at least 80% sequence homology with the amino acid sequence shown in SEQ ID No. 7, SEQ ID No. 15, SEQ ID No. 23, SEQ ID No. 31, SEQ ID No. 39, SEQ ID No. 47 or SEQ ID No. 55 in the sequence listing; and the amino acid sequence of the light chain CDR3 is as shown in an amino acid sequence which has at least 80% sequence homology with the amino acid sequence shown in SEQ ID No. 8, SEQ ID No. 16, SEQ ID No. 24, SEQ ID No. 32, SEQ ID No. 40, SEQ ID No. 48 or SEQ ID No. 56 in the sequence listing.

Preferably, the amino acid sequence of the heavy chain CDR1 is as shown in SEQ ID No. 2 in the sequence listing, the amino acid sequence of the heavy chain CDR2 is as shown in SEQ ID No. 3 in the sequence listing, and the amino acid sequence of the heavy chain CDR3 is as shown in SEQ ID No. 4 in the sequence listing; the amino acid sequence of the heavy chain CDR1 is as shown in SEQ ID No. 10 in the sequence listing, the amino acid sequence of the heavy chain CDR2 is as shown in SEQ ID No. 11 in the sequence listing, and the amino acid sequence of the heavy chain CDR3 is as shown in SEQ ID No. 12 in the sequence listing; the amino acid sequence of the heavy chain CDR1 is as shown in SEQ ID No. 18 in the sequence listing, the amino acid sequence of the heavy chain CDR2 is as shown in SEQ ID No. 19 in the sequence listing, and the amino acid sequence of the heavy chain CDR3 is as shown in SEQ ID No. 20 in the sequence listing; the amino acid sequence of the heavy chain CDR1 is as shown in SEQ ID No. 26 in the sequence listing, the amino acid sequence of the heavy chain CDR2 is as shown in SEQ ID No. 27 in the sequence listing, and the amino acid sequence of the heavy chain CDR3 is as shown in SEQ ID No. 28 in the sequence listing; the amino acid sequence of the heavy chain CDR1 is as shown in SEQ ID No. 34 in the sequence listing, the amino acid sequence of the heavy chain CDR2 is as shown in SEQ ID No. 35 in the sequence listing, and the amino acid sequence of the heavy chain CDR3 is as shown in SEQ ID No. 36 in the sequence listing; the amino acid sequence of the heavy chain CDR1 is as shown in SEQ ID No. 42 in the sequence listing, the amino acid sequence of the heavy chain CDR2 is as shown in SEQ ID No. 43 in the sequence listing, and the amino acid sequence of the heavy chain CDR3 is as shown in SEQ ID No. 44 in the sequence listing; the amino acid sequence of the heavy chain CDR1 is as shown in SEQ ID No. 50 in the sequence listing, the amino acid sequence of the heavy chain CDR2 is as shown in SEQ ID No. 51 in the sequence listing, and the amino acid sequence of the heavy chain CDR3 is as shown in SEQ ID No. 52 in the sequence listing; the amino acid sequence of the light chain CDR1 is as shown in SEQ ID No. 6 in the sequence listing, the amino acid sequence of the light chain CDR2 is as shown in SEQ ID No. 7 in the sequence listing, and the amino acid sequence of the light chain CDR3 is as shown in SEQ ID No. 8 in the sequence listing; the amino acid sequence of the light chain CDR1 is as shown in SEQ ID No. 14 in the sequence listing, the amino acid sequence of the light chain CDR2 is as shown in SEQ ID No. 15 in the sequence listing, and the amino acid sequence of the light chain CDR3 is as shown in SEQ ID No. 16 in the sequence listing; the amino acid sequence of the light chain CDR1 is as shown in SEQ ID No. 22 in the sequence listing, the amino acid sequence of the light chain CDR2 is as shown in SEQ ID No. 23 in the sequence listing, and the amino acid sequence of the light chain CDR3 is as shown in SEQ ID No. 24 in the sequence listing; the amino acid sequence of the light chain CDR1 is as shown in SEQ ID No. 30 in the sequence listing, the amino acid sequence of the light chain CDR2 is as shown in SEQ ID No. 31 in the sequence listing, and the amino acid sequence of the light chain CDR3 is as shown in SEQ ID No. 32 in the sequence listing; the amino acid sequence of the light chain CDR1 is as shown in SEQ ID No. 38 in the sequence listing, the amino acid sequence of the light chain CDR2 is as shown in SEQ ID No. 39 in the sequence listing, and the amino acid sequence of the light chain CDR3 is as shown in SEQ ID No. 40 in the sequence listing; the amino acid sequence of the light chain CDR1 is as shown in SEQ ID No. 46 in the sequence listing, the amino acid sequence of the light chain CDR2 is as shown in SEQ ID No. 47 in the sequence listing, and the amino acid sequence of the light chain CDR3 is as shown in SEQ ID No. 48 in the sequence listing; or the amino acid sequence of the light chain CDR1 is as shown in SEQ ID No. 54 in the sequence listing, the amino acid sequence of the light chain CDR2 is as shown in SEQ ID No. 55 in the sequence listing, and the amino acid sequence of the light chain CDR3 is as shown in SEQ ID No. 56 in the sequence listing.

The present invention provides an isolated protein comprising the heavy chain variable region of an anti-C5aR antibody and/or the light chain variable region of an anti-C5aR antibody, wherein the amino acid sequence of the heavy chain variable region is as shown in SEQ ID No. 1, SEQ ID No. 9, SEQ ID No. 17, SEQ ID No. 25, SEQ ID No. 33, SEQ ID No. 41 or SEQ ID No. 49 in the sequence listing; the amino acid sequence of the light chain variable region is as shown in SEQ ID No. 5, SEQ ID No. 13, SEQ ID No. 21, SEQ ID No. 29, SEQ ID No. 37, SEQ ID No. 45 or SEQ ID No. 53 in the sequence listing.

Preferably, the amino acid sequence of the heavy chain variable region is as shown in SEQ ID No. 1 in the sequence listing, and the amino acid sequence of the light chain variable region is as shown in SEQ ID No. 5 in the sequence listing; the amino acid sequence of the heavy chain variable region is as shown in SEQ ID No. 9 in the sequence listing, and the amino acid sequence of the light chain variable region is as shown in SEQ ID No. 13 in the sequence listing; the amino acid sequence of the heavy chain variable region is as shown in SEQ ID No. 17 in the sequence listing, and the amino acid sequence of the light chain variable region is as shown in SEQ ID No. 21 in the sequence listing; the amino acid sequence of the heavy chain variable region is as shown in SEQ ID No. 25 in the sequence listing, and the amino acid sequence of the light chain variable region is as shown in SEQ ID No. 29 in the sequence listing; the amino acid sequence of the heavy chain variable region is as shown in SEQ ID No. 33 in the sequence listing, and the amino acid sequence of the light chain variable region is as shown in SEQ ID No. 37 in the sequence listing; the amino acid sequence of the heavy chain variable region is as shown in SEQ ID No. 41 in the sequence listing, and the amino acid sequence of the light chain variable region is as shown in SEQ ID No. 45 in the sequence listing; or the amino acid sequence of the heavy chain variable region is as shown in SEQ ID No. 49 in the sequence listing, and the amino acid sequence of the light chain variable region is as shown in SEQ ID No. 53 in the sequence listing.

In summary, the numbering of the above-mentioned amino acid sequences is shown in table 1:

TABLE 1
Numbers of protein sequence of anti-C5aR antibody

| | Heavy chain protein | | | | Light chain protein | | | |
|---|---|---|---|---|---|---|---|---|
| Clone | Variable | CD | CD | CD | Variable | CD | CD | CD |
| numbers | region | R1 | R2 | R3 | region | R1 | R2 | R3 |
| 5F8E2C11 | 1 | 2 | 3 | 4 | 5 | 6 | 7 | 8 |
| 42B5G7D1 | 9 | 10 | 11 | 12 | 13 | 14 | 15 | 16 |
| 43E8F5B6 | 17 | 18 | 19 | 20 | 21 | 22 | 23 | 24 |
| 46H2A11C7 | 25 | 26 | 27 | 28 | 29 | 30 | 31 | 32 |
| 2A12B2B2 | 33 | 34 | 35 | 36 | 37 | 38 | 39 | 40 |
| 8B5D1A9 | 41 | 42 | 43 | 44 | 45 | 46 | 47 | 48 |
| 9D5Al2G7 | 49 | 50 | 51 | 52 | 53 | 54 | 55 | 56 |

The numbers in table 1 are the numbers “SEQ ID Nos.” in the sequence listing, for example, the amino acid sequence of the heavy chain protein variable region of 5F8E2C11 is SEQ ID No. 1 in the sequence listing, and the amino acid sequence of CDR1 domain in the heavy chain protein variable region of 5F8E2C11 is SEQ ID No. 2 in the sequence listing.

Preferably, the protein further comprises an antibody heavy chain constant region and/or antibody light chain constant region, and the antibody heavy chain constant region is conventional in the art, preferably is a mouse antibody heavy chain constant region; the antibody light chain constant region is conventional in the art, preferably is a mouse antibody light chain constant region.

The proteins are conventional proteins in the art, preferably is an anti-C5aR antibody, more preferably are one or more of full-length antibody proteins, antigen-antibody binding domain protein fragments, bispecific antibodies, polyspecific antibodies, single chain antibody fragments (scFv), single domain antibodies (sdAb) and single-domain antibodies, and are monoclonal antibodies or polyclonal antibodies which are prepared from the above-mentioned antibodies. The monoclonal antibodies can be developed by many ways and techniques, including hybridoma technique, phage display technology, single lymphocyte gene cloning technology etc., and monoclonal antibodies are mainly prepared from wild type or transgenic mice by hybridoma technique.

The full-length antibody proteins are conventional full-length antibody proteins in the art, which comprise a heavy chain variable region, light chain variable region, heavy chain constant region and light chain constant region. The heavy chain variable region and light chain variable region of the proteins can also constitute a complete human full-length antibody protein with a human heavy chain constant region and human light chain constant region. Preferably, the full-length antibody protein is IgG1, IgG2, IgG3 or IgG4.

The single chain antibody fragments are conventional single chain antibody fragments in the art, which comprise a heavy chain variable region, a light chain variable region and a short peptide with 15-20 amino acids.

The antigen-antibody binding domain protein fragments are conventional antigen-antibody binding domain protein fragments in the art, which comprise a light chain variable region, a light chain constant region and Fd fragment of a heavy chain constant region. Preferably, the antigen-antibody binding domain protein fragments are Fab and F(ab′).

The single domain antibodies are conventional single domain antibodies in the art, which comprise a heavy chain variable region and heavy chain constant region.

The single-domain antibodies are conventional single-domain antibodies in the art, which only comprise a heavy chain variable region.

In the document, the method for preparing the protein is a conventional preparation method in the art. Preferably, the preparation method is: obtaining the protein by separation from an expression transformant recombinantly expressing the protein, or obtaining the protein by artificial synthesis of the protein sequence. The method for obtaining the protein by separation from an expression transformant recombinantly expressing the protein is preferably as follows: cloning the nucleic acid molecule encoding the protein and having a point mutation into a recombinant vector, transforming the obtained recombinant vector into a transformant to obtain a recombinant expression transformant, and culturing the obtained recombinant expression transformant, finally obtaining the protein by separation and purification.

The present invention also provides a nucleic acid encoding the above-mentioned protein.

Preferably, the nucleic acid encoding the heavy chain variable region is as shown in SEQ ID No. 57 in the sequence listing, SEQ ID No. 59 in the sequence listing, SEQ ID No. 61 in the sequence listing, SEQ ID No. 63 in the sequence listing, SEQ ID No. 65 in the sequence listing, SEQ ID No. 67 in the sequence listing or SEQ ID No. 69 in the sequence listing; and/or the nucleotide sequence of the nucleic acid encoding the light chain variable region is as shown in SEQ ID No. 58 in the sequence listing, SEQ ID No. 60 in the sequence listing, SEQ ID No. 62 in the sequence listing, SEQ ID No. 64 in the sequence listing, SEQ ID No. 66 in the sequence listing, SEQ ID No. 68 in the sequence listing or SEQ ID No. 70 in the sequence listing.

Preferably, the nucleic acid encoding the heavy chain variable region is as shown in SEQ ID No. 57 in the sequence listing, and the nucleic acid encoding the light chain variable region is as shown in SEQ ID No. 58 in the sequence listing; the nucleic acid encoding the heavy chain variable region is as shown in SEQ ID No. 59 in the sequence listing, and the nucleic acid encoding the light chain variable region is as shown in SEQ ID No. 60 in the sequence listing; the nucleic acid encoding the heavy chain variable region is as shown in SEQ ID No. 61 in the sequence listing, and the nucleic acid encoding the light chain variable region is as shown in SEQ ID No. 62 in the sequence listing; the nucleic acid encoding the heavy chain variable region is as shown in SEQ ID No. 63 in the sequence listing, and the nucleic acid encoding the light chain variable region is as shown in SEQ ID No. 64 in the sequence listing; the nucleic acid encoding the heavy chain variable region is as shown in SEQ ID No. 65 in the sequence listing, and the nucleic acid encoding the light chain variable region is as shown in SEQ ID No. 66 in the sequence listing; the nucleic acid encoding the heavy chain variable region is as shown in SEQ ID No. 67 in the sequence listing, and the nucleic acid encoding the light chain variable region is as shown in SEQ ID No. 68 in the sequence listing; or the nucleic acid encoding the heavy chain variable region is as shown in SEQ ID No. 69 in the sequence listing, and the nucleic acid encoding the light chain variable region is as shown in SEQ ID No. 70 in the sequence listing.

The numbering of the above-mentioned nucleotide sequences are shown in table 2:

TABLE 2
Numbers of gene sequence of anti-C5aR antibody

| | Heavy chain proteins | Light chain proteins |
|---|---|---|
| Clone numbers | variable region | Variable region |
| 5F8E2C11 | 57 | 58 |
| 42B5G7D1 | 59 | 60 |
| 43E8F5B6 | 61 | 62 |
| 46H2A11C7 | 63 | 64 |
| 2A12B2B2 | 65 | 66 |
| 8B5D1A9 | 67 | 68 |
| 9D5A12G7 | 69 | 70 |

The numbers in table 2 are the numbers “SEQ ID Nos.” in the sequence listing, for example, the nucleotide sequence encoding the heavy chain protein variable region of 5F8E2C11 is SEQ ID No. 33 in the sequence listing.

The nucleotide sequence encoding CDR1 in the heavy chain protein variable region of 5F8E2C11 is positions 76 to 105 of SEQ ID No. 57 in the sequence listing;

the nucleotide sequence encoding CDR2 in the heavy chain protein variable region of 5F8E2C11 is positions 148 to 195 of SEQ ID No. 57 in the sequence listing;

the nucleotide sequence encoding CDR3 in the heavy chain protein variable region of 5F8E2C11 is positions 292 to 321 of SEQ ID No. 57 in the sequence listing;

the nucleotide sequence encoding CDR1 in the light chain protein variable region of 5F8E2C11 is positions 70 to 120 of SEQ ID No. 58 in the sequence listing;

the nucleotide sequence encoding CDR2 in the light chain protein variable region of 5F8E2C11 is positions 166 to 186 of SEQ ID No. 58 in the sequence listing;

the nucleotide sequence encoding CDR3 in the light chain protein variable region of 5F8E2C11 is positions 283 to 306 of SEQ ID No. 58 in the sequence listing;

the nucleotide sequence encoding CDR1 in the heavy chain protein variable region of 42B5G7D1 is positions 76 to 105 of SEQ ID No. 59 in the sequence listing;

the nucleotide sequence encoding CDR2 in the heavy chain protein variable region of 42B5G7D1 is positions 148 to 198 of SEQ ID No. 59 in the sequence listing;

the nucleotide sequence encoding CDR3 in the heavy chain protein variable region of 42B5G7D1 is positions 295 to 336 of SEQ ID No. 59 in the sequence listing;

the nucleotide sequence encoding CDR1 in the light chain protein variable region of 42B5G7D1 is positions 70 to 117 of SEQ ID No. 60 in the sequence listing;

the nucleotide sequence encoding CDR2 in the light chain protein variable region of 42B5G7D1 is positions 163 to 183 of SEQ ID No. 60 in the sequence listing;

the nucleotide sequence encoding CDR3 in the light chain protein variable region of 42B5G7D1 is positions 280 to 306 of SEQ ID No. 60 in the sequence listing;

the nucleotide sequence encoding CDR1 in the heavy chain protein variable region of 43E8F5B6 is positions 76 to 105 of SEQ ID No. 61 in the sequence listing;

the nucleotide sequence encoding CDR2 in the heavy chain protein variable region of 43E8F5B6 is positions 148 to 198 of SEQ ID No. 61 in the sequence listing;

the nucleotide sequence encoding CDR3 in the heavy chain protein variable region of 43E8F5B6 is positions 295 to 339 of SEQ ID No. 61 in the sequence listing;

the nucleotide sequence encoding CDR1 in the light chain protein variable region of 43E8F5B6 is positions 70 to 105 of SEQ ID No. 62 in the sequence listing;

the nucleotide sequence encoding CDR2 in the light chain protein variable region of 43E8F5B6 is positions 151 to 171 of SEQ ID No. 62 in the sequence listing;

the nucleotide sequence encoding CDR3 in the light chain protein variable region of 43E8F5B6 is positions 268 to 294 of SEQ ID No. 62 in the sequence listing;

the nucleotide sequence encoding CDR1 in the heavy chain protein variable region of 46H2A11C7 is positions 76 to 105 of SEQ ID No. 63 in the sequence listing;

the nucleotide sequence encoding CDR2 in the heavy chain protein variable region of 46H2A11C7 is positions 148 to 198 of SEQ ID No. 63 in the sequence listing;

the nucleotide sequence encoding CDR3 in the heavy chain protein variable region of 46H2A11C7 is positions 295 to 306 of SEQ ID No. 63 in the sequence listing;

the nucleotide sequence encoding CDR1 in the light chain protein variable region of 46H2A11C7 is positions 70 to 117 of SEQ ID No. 64 in the sequence listing;

the nucleotide sequence encoding CDR2 in the light chain protein variable region of 46H2A11C7 is positions 163 to 183 of SEQ ID No. 64 in the sequence listing;

the nucleotide sequence encoding CDR3 in the light chain protein variable region of 46H2A11C7 is positions 280 to 306 of SEQ ID No. 64 in the sequence listing;

the nucleotide sequence encoding CDR1 in the heavy chain protein variable region of 2A12B2B2 is positions 76 to 105 of SEQ ID No. 65 in the sequence listing;

the nucleotide sequence encoding CDR2 in the heavy chain protein variable region of 2A12B2B2 is positions 148 to 198 of SEQ ID No. 65 in the sequence listing;

the nucleotide sequence encoding CDR3 in the heavy chain protein variable region of 2A12B2B2 is positions 295 to 303 of SEQ ID No. 65 in the sequence listing;

the nucleotide sequence encoding CDR1 in the light chain protein variable region of 2A12B2B2 is positions 70 to 102 of SEQ ID No. 66 in the sequence listing;

the nucleotide sequence encoding CDR2 in the light chain protein variable region of 2A12B2B2 is positions 148 to 168 of SEQ ID No. 66 in the sequence listing;

the nucleotide sequence encoding CDR3 in the light chain protein variable region of 2A12B2B2 is positions 265 to 291 of SEQ ID No. 66 in the sequence listing;

the nucleotide sequence encoding CDR1 in the heavy chain protein variable region of 8B5D1A9 is positions 76 to 105 of SEQ ID No. 67 in the sequence listing;

the nucleotide sequence encoding CDR2 in the heavy chain protein variable region of 8B5D1A9 is positions 148 to 198 of SEQ ID No. 67 in the sequence listing;

the nucleotide sequence encoding CDR3 in the heavy chain protein variable region of 8B5D1A9 is positions 295 to 324 of SEQ ID No. 67 in the sequence listing;

the nucleotide sequence encoding CDR1 in the light chain protein variable region of 8B5D1A9 is positions 70 to 120 of SEQ ID No. 68 in the sequence listing;

the nucleotide sequence encoding CDR2 in the light chain protein variable region of 8B5D1A9 is positions 166 to 189 of SEQ ID No. 68 in the sequence listing;

the nucleotide sequence encoding CDR3 in the light chain protein variable region of 8B5D1A9 is positions 286 to 312 of SEQ ID No. 68 in the sequence listing;

the nucleotide sequence encoding CDR1 in the heavy chain protein variable region of 9D5A12G7 is positions 76 to 105 of SEQ ID No. 69 in the sequence listing;

the nucleotide sequence encoding CDR2 in the heavy chain protein variable region of 9D5A12G7 is positions 148 to 195 of SEQ ID No. 69 in the sequence listing;

the nucleotide sequence encoding CDR3 in the heavy chain protein variable region of 9D5A12G7 is positions 292 to 321 of SEQ ID No. 69 in the sequence listing;

the nucleotide sequence encoding CDR1 in the light chain protein variable region of 9D5A12G7 is positions 70 to 120 of SEQ ID No. 70 in the sequence listing;

the nucleotide sequence encoding CDR2 in the light chain protein variable region of 9D5A12G7 is positions 166 to 186 of SEQ ID No. 70 in the sequence listing;

and the nucleotide sequence encoding CDR3 in the light chain protein variable region of 9D5A12G7 is positions 283 to 306 of SEQ ID No. 70 in the sequence listing.

The method for preparing the nucleic acid is a conventional preparation method in the art, preferably, the method comprises the following steps: obtaining the nucleic acid molecule encoding the above-mentioned protein by gene cloning technology, or obtaining the nucleic acid molecule encoding the above-mentioned protein by artificial complete sequence synthetic method.

It is known to those skilled in the art that the base sequence encoding the amino acid sequence of the above-mentioned protein can be appropriately introduced a substitution, deletion, alteration, insertion or addition to provide a homolog of a polynucleotide. The homolog of a polynucleotide in the present invention can be prepared by the substitution, deletion or addition of one or more bases of the gene encoding the protein sequence in the range of maintaining the antibody activity.

The present invention also provides a recombinant expression vector comprising the nucleic acid,

wherein the recombinant expression vector can be obtained by a conventional method in the art, namely: it is constructed by ligating the nucleic acid molecule of the present invention to various expression vectors. The expression vectors are various conventional vectors in the art, as long as it can carry the above-mentioned nucleic acid molecule. Preferably, the vectors include various plasmids, cosmids, phage or viral vectors etc.

The present invention also provides a recombinant expression transformant comprising the above-mentioned recombinant expression vector,

wherein the method for preparing the recombinant expression transformant is a conventional preparation method in the art, preferably, it is obtained by transforming the above-mentioned recombinant expression vector into a host cell. The host cells are various conventional host cells in the art, as long as it meets the following requirements: in the host cells, the above-mentioned recombinant expression vector is stably replicated by oneself, and the carried nucleic acid can be expressed effectively. Preferably, the host cell is E. coli TG1 or BL21 cells (expressing single chain antibody fragments or Fab antibody), or CHO-K1 cell (expressing full-length IgG antibody). The above-mentioned recombinant expression plasmid is transformed into a host cell, then a preferred recombinant expression transformant of the present invention can be obtained, wherein the transformation method is a conventional transformation method in the art, preferably is a chemical transformation method, a heat shock method or an electroporation method.

The present invention provides a method for preparing an anti-C5aR antibody, comprising the following steps: culturing the above-mentioned recombinant expression transformant, and obtaining the anti-C5aR antibody from the culture.

The present invention provides a method for detecting a cell overexpressing C5aR protein, comprising the following steps: contacting the above-mentioned protein with a sample to be detected in vitro, then detecting the binding of the above-mentioned protein and the sample to be detected.

The meaning of the overexpression is conventional in the art and refers to that the overexpression of the RNA or protein of C5aR protein in the sample to be detected (because of increased transcription, post-transcriptional processing, translation, post-translation processing and protein degradation changes), and the local overexpression and increased functional activity (such as in the case that the enzymatic hydrolysis of the substrate is increased) caused by changes in protein transport modes (increased nuclear localization). Preferably, overexpression also means that the RNA or protein expression level of C5aR protein is increased by 50%, 60%, 70%, 80%, 90% or more than that of a control sample or normal cells.

The detection mode of the binding is a conventional detection mode in the art, preferably is FACS detection.

The present invention provides the use of the above-mentioned protein in the preparation of a drug.

Preferably, the drug is a drug for treating inflammation, blood vessel diseases or nervous system diseases.

The present invention also provides a pharmaceutical composition, of which active ingredients comprise the above-mentioned protein.

Preferably, the pharmaceutical composition is a pharmaceutical composition for treating inflammation, blood vessel diseases or nervous system diseases.

Preferably, the administration route of the pharmaceutical composition of the present invention is injection administration or oral administration. Preferably, the injection administration comprises the routes such as intravenous injection, intramuscular injection, intraperitoneal injection, intracutaneous injection or subcutaneous injection. The pharmaceutical composition is in various conventional dosage forms in the art, preferably, in the forms of solid, semisolid or liquid; and can be an aqueous solution, non-aqueous solution or suspension; more preferably, is a tablet, capsule, granule, injection or infusion etc.

Preferably, the pharmaceutical composition of the present invention also comprises one or more pharmaceutically acceptable carriers. The pharmaceutically acceptable carrier is a conventional pharmaceutically acceptable carrier in the art, and can be any suitable physiologically or pharmaceutically acceptable pharmaceutical auxiliary materials. The pharmaceutical auxiliary material is a conventional pharmaceutical auxiliary material in the art, preferably, comprising a pharmaceutically acceptable excipient, filler or diluent etc. More preferably, the pharmaceutical composition comprises 0.01-99.99% of the above-mentioned protein and 0.01-99.99% of the pharmaceutically acceptable carrier, and the percentage is a mass percentage accounting for the pharmaceutical composition.

Preferably, the administration amount of the pharmaceutical composition is an effective amount, and the effective amount is an amount that can alleviate or delay the progress of diseases and degenerative or traumatic conditions. The effective amount can be determined on individual basis, and will be based partly on considerations of the symptom to be treated and the searched result. The effective amount can be determined by those skilled in the art using the above-mentioned factors such as individual basis and using experiments which are not beyond conventional ones.

The present invention provides the use of the above-mentioned protein in the treatment of inflammation, blood vessel diseases or nervous system diseases.

The present invention provides the use of the above-mentioned pharmaceutical composition in the treatment of inflammation, blood vessel diseases or nervous system diseases.

On the basis of meeting common knowledge in the art, the above-mentioned various preferred conditions can be combined in any form, such that various preferred examples of the present invention are obtained.

Reagents and raw materials used in the present invention are all commercially available.

The present invention has the following positive improvement effects: the protein of the present invention is an anti-C5aR antibody, wherein the anti-C5aR antibody has a high affinity (the affinity KD<1*10⁻⁸ M) to C5aR protein, can effectively inhibit or block the binding of C5aR to C5a and significantly reduce a calcium flow signal, thus downregulating or switching off a corresponding signaling pathway, stopping C5a directed migration of a neutrophil in vitro and inhibiting chemotaxis of the neutrophil. Therefore, the anti-C5aR antibody can be used the preparation of a drug for treating inflammation, blood vessel diseases or nervous system diseases.

### BRIEF DESCRIPTION OF THE DRAWINGS

FIG. 1 is the results of FACS screening and detection on HEK293 cells transfected by C5aR protein.

FIG. 2 is the detection of the serum antibody titer of mice after being immunized by C5aR polypeptide via ELISA.

FIG. 3 is the detection of the binding of anti-C5aR antibody and specific epitopes of C5aR via ELISA.

FIG. 4 is the detection of the binding reaction between anti-C5aR antibody and CHOK1-hC5aR via FACS.

FIG. 5 is the detection of the blocking of C5a-mediated calcium flow signal by anti-C5aR antibody via FLIPR.

FIG. 6 is the detection of the blocking of C5a-induced cell migration by anti-C5aR antibody via human neutrophil chemotaxis experiments.

### DETAILED DESCRIPTION OF THE PREFERRED EMBODIMENT

The following examples further illustrate the present invention, but the present invention is not limited thereto. Experimental methods with specific conditions are not indicated in the following examples, but can be chosen according to conventional methods and conditions or commodity instructions.

The room temperature in the examples is a conventional room temperature in the art, generally is 10-30° C.

### Example 1

### Preparation of Anti-C5aR Antibody

### (I) Preparation of Immunogen A

C5aR is a seven-transmembrane protein, of which the extracellular domain comprises 1 N-terminus and 3 extracellular loops. 5 polypeptides were designed for the N-terminus and 3 extracellular loops of C5aR protein, the specific sequence is shown in table 3. The polypeptides shown in table 3 were synthesized by GL Biochem (Shanghai) Co., Ltd. These polypeptides were coupled to keyhole limpet hemocyanin (KLH), then immunogen A was obtained. The coupling method is shown in “Development and Application of Indirect ELISA for Detecting PRRS Antibody”, [J]. Chinese Journal of Veterinary Science and Technology, 2005 (6).

TABLE 3
Polypeptide sequence of immunogen A

| No. | Extracellular domain | Sequence |
|---|---|---|
| C5aR polypeptide-01 | C5aR extracellular N- | See SEQ ID No. 43 in the |
| | terminus-1 | sequence listing |
| C5aR polypeptide-02 | C5aR extracellular N- | See SEQ ID No. 44 in the |
| | terminus-2 | sequence listing |
| C5aR polypeptide-03 | C5aR extracellular | See SEQ ID No. 45 in the |
| | loop-1 | sequence listing |
| C5aR polypeptide-04 | C5aR extracellular | See SEQ ID No. 46 in the |
| | loop-2 | sequence listing |
| C5aR polypeptide-05 | C5aR extracellular | See SEQ ID No. 47 in the |
| | loop-3 | sequence listing |

### (II) Preparation of Immunogen B

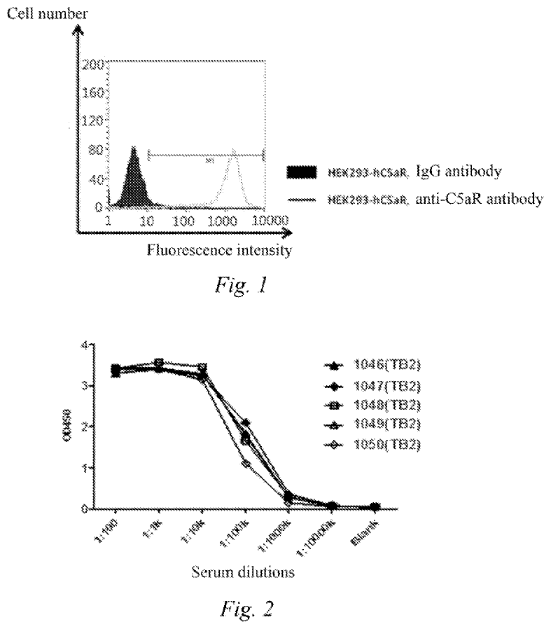

A stable cell line for immunogen B uses the lentivirus infection method. The nucleotide sequence encoding the full-length amino acid sequence of human C5aR (as shown in SEQ ID No. 72 of the sequence listing) was cloned into a lentiviral vector pLVX-IRES vector (purchased from Clontech) and a plasmid was prepared. By using liposome transfection method, the plasmid was transfected (using X-treme GENE HP DNA Transfection Reagent, purchased from Roche, Cat #06 366 236 001, and operating according to the instructions) into HEK293 cell line for virus packaging, then the virus suspension was collected for infecting HEK293 cell line and CHOK1 cell line (both purchased from Invitrogen). The infection method was as follows: spreading cells of HEK293 cell line and CHOK1 cell line onto complete medium containing 10% (w/w) fetal bovine serum in a 6-well plate at a concentration of 1 e⁵ cells/well, wherein the complete medium for HEK293 was DMEM medium (purchased from Gibco), and the complete medium for CHOK1 was Ham's F-12 Nutrient Mixture medium (purchased from Gibco), and then placing the 6-well plate into a incubator under the condition of 37° C. and 5% (v/v) CO₂ and cultivating overnight. The next day, the medium was discarded, the virus suspension was added at 1 ml/well, after being incubated overnight, the virus infection fluid was discarded. Then 2 ml DMEM medium (purchased from Invitrogen) containing 800 μg/mL Hygromycin B and 10% (w/w) fetal bovine serum was added for a selective culture of 2 weeks. The culture was subcloned in a 96-well culture plate by using limiting dilution, and was placed under the conditions of 37° C. and 5% (v/v) CO₂ and cultured, about 2 weeks later, a part of monoclonal wells were selected and amplified into a 6-well plate. The amplified clones were screened by flow cytometry analysis using known anti-C5aR antibody (purchased from Abcam). A monoclonal cell line with a good growth vigour and high fluorescence intensity was selected for further amplification culture, and same was cryopreserved in liquid nitrogen, then immunogen B was obtained. The specific selection results are shown in table 4 and FIG. 1. The positive cells (%) in table 4 refers to the percentage of positive cells accounting for the total cell number. Table 4 shows that a series of HEK293 cell lines with positive C5aR expression were prepared, and therefore immunogen B was obtained.

TABLE 4
Results of FACS screening and detection
on HEK293 cells transfected by C5aR protein

| | | anti-C5aR antibody | | IgG subtype control | |
|---|---|---|---|---|---|
| | | | Mean | | Mean |
| | Clone numbers of | Positive | fluorescence | Positive | fluorescence |
| No. | transfected cells | cells (%) | intensity | cells (%) | intensity |
| 1 | 293F-hC5aR 1C4 | 99.18 | 1927.20 | 0.90 | 4.46 |
| 2 | 293F-hC5aR 1C2 | 99.21 | 2260.48 | 2.40 | 3.21 |
| 3 | 293F-hC5aR 1F2 | 99.29 | 2655.42 | 1.70 | 4.75 |

### (III) Preparation of Immunogen C

The full-length amino acid sequence cDNA of human C5aR (as shown in SEQ ID No. 71 of the sequence listing) was cloned into pCDNA3.1 vector (purchased from Invitrogen), and coated onto 1.0 μm colloidal gold bullet (purchased from Bio-rad), and was immunized by using Helios Gene Gun System (Bio-rad, Cat #165-2431), then immunogen C was obtained. During the preparation, the methods of coating onto 1.0 m colloidal gold bullet and immunization were made according to instructions of Helios Gene Gun System.

A. For being immunized with immunogen A, 6-8 weeks old Balb/c and SJL mice (purchased from Shanghai SLAC Laboratory Animal Co., Ltd) were used, and the mice were raised under SPF conditions. For the primary immunization, immunogen A was emulsified with Freund's complete adjuvant, then was intraperitoneally injected at 0.25 milliliters, i.e. 50 micrograms of immunogen A was injected into each mouse. For the booster immunization, immunogen A was emulsified with Freund's incomplete adjuvant, then was intraperitoneally injected at 0.25 milliliters, i.e. 50 micrograms of immunogen A was injected into each mouse. The interval between the primary immunization and first booster immunization was 2 weeks, and then the interval between one subsequent booster immunization and the next booster immunization was 3 weeks. Blood was collected 1 week after each booster immunization, the antibody titer and specificity of immunogen A in the serum were detected by using ELISA and FACS. The results are shown in FIG. 2 and table 5. Table 5 shows that the serum of mice immunized by the C5aR extracellular region polypeptide binds to the immunogen in varying degrees and exhibits antigen-antibody reaction, wherein the highest dilution is about 10⁶. In the table, the blank control is 1% (w/w) BSA, the batch refers to the serum of mice at day 7 after the third booster immunization, and the data in the table is OD_{450 nm} values.

TABLE 5
Detection of the serum antibody titer of mice after being
immunized by C5aR polypeptide via ELISA

| | Serum dilutions | | | | | | |
|---|---|---|---|---|---|---|---|
| OD_{450 nm} | | | | | | | Blank |
| Batch | 1 : 100 | 1 : 10³ | 1 : 10⁴ | 1 : 10⁵ | 1 : 10⁶ | 1 : 10⁷ | control |
| 1046 | 3.43 | 3.42 | 3.29 | 1.80 | 0.28 | 0.07 | 0.05 |
| (TB2) | | | | | | | |
| 1047 | 3.31 | 3.40 | 3.24 | 2.10 | 0.36 | 0.08 | 0.05 |
| (TB2) | | | | | | | |
| 1048 | 3.42 | 3.57 | 3.45 | 1.66 | 0.29 | 0.07 | 0.05 |
| (TB2) | | | | | | | |
| 1049 | 3.29 | 3.42 | 3.27 | 1.84 | 0.28 | 0.07 | 0.05 |
| (TB2) | | | | | | | |
| 1050 | 3.41 | 3.40 | 3.15 | 1.10 | 0.15 | 0.06 | 0.05 |
| (TB2) | | | | | | | |

B. For being immunized with immunogen B, 6-8 weeks old Balb/c and SJL mice (purchased from Shanghai SLAC Laboratory Animal Co., Ltd) were used, and the mice were raised under SPF conditions. pIRES plasmid containing the nucleotide sequence encoding the full-length amino acid sequence of human C5aR [see example 1, step (II)] was transfected into HEK293 cell line, and a stable HEK293 cell line containing human C5aR was obtained (using X-treme GENE HP DNA Transfection Reagent, purchased from Roche, Cat #06366236001, and operating according to the instructions). The cells were amplified and cultured in a T-75 cell culture flask to 90% confluence, the medium was sucked out, and the cells were washed twice with DMEM basic medium twice, then were treated with an enzyme-free cell dissociation fluid (purchased from Invitrogen) at 37° C. until the cells can be fallen off from the wall of the culture dish, and the cells were collected. The cells were washed twice with DMEM basic medium, and were counted, then were diluted to 2×10⁷ cells per milliliter with phosphate buffer (pH 7.2). For each immunization, 0.5 milliliter cell suspension was intraperitoneally injected into each mouse. The interval between the first immunization and second immunization was 2 weeks, and then the interval between one subsequent immunization and the next immunization was 3 weeks. In addition to the first immunization, blood was collected 1 week after each immunization, and the antibody titer and specificity in the serum were detected by using FACS. After the second booster immunization, the serum antibody titer which was detected by FACS reached at least 1:1000.

C. For being immunized with immunogen C, 6-8 weeks old Balb/c and SJL mice (purchased from Shanghai SLAC Laboratory Animal Co., Ltd) were used, and the mice were raised under SPF conditions. All mice were immunized intraperitoneally 4 times by using Helios Gene Gun System, 4 shots each time, and 1.0 microgram cDNA for each shot. The interval between the primary immunization and first booster immunization was 2 weeks, and then the interval between one subsequent booster immunization and the next booster immunization was 3 weeks. Blood was collected 1 week after each booster immunization, the antibody titer in the serum were detected by using ELISA or FACS. After the second booster immunization, the serum antibody titer which was detected by FACS reached at least 1:1000, and the titer which was detected by ELISA reached at least 1:10000.

The immunizations generally used immunogens A-C, and in most mice after being immunized 3 times, all the titer which was detected by FACS can reach at least 1:1000.

3-5 days before cell fusion, the selected mice were subjected to the last booster immunization, wherein the mice immunized with immunogen A were finally immunized with the corresponding polypeptides as shown in table 3; the mice immunized with immunogens B and C were finally immunized with the stable HEK293 cell line expressing human C5aR. 3-5 days later, the mice were sacrificed and spleen cells were collected. NH₄OH was added to a final concentration of 1% (w/w), red blood cells incorporated in the spleen cells were lysed and a spleen cell suspension was obtained. The cells were washed with DMEM basic medium and centrifuged at 1000 rpm for 3 times, then were mixed at a 5:1 ratio of living cell number with mouse myeloma cells SP2/0 (purchased from ATCC), then were subjected to cell fusion by using an efficient electrical fusion method (see METHODS IN ENZYMOLOGY, VOL. 220). The fused cells were diluted into DMEM medium containing 20% fetal bovine serum and 1×HAT, wherein the percentage is the mass percentage. Then the cells were added into a 96-well cell culture plate at 1×10⁵ cells/200 microliter/well, and the plate was placed into a incubator with 5% CO₂ and at 37° C., wherein the percentage is the volume percentage. 14 days later, the supernatant in the cell fusion plate was screened by Acumen (microwell plate cell detection method), the positive clones with MFI value >100 in Acumen were amplified into a 24-well plate, and were amplified and cultured in DMEM medium containing 10% (w/w) HT fetal bovine serum under the conditions of 37° C. and 5% (v/v) CO₂. After 3 day culture, the amplified and cultured culture solution in the 24-well plate was centrifuged, the supernatant was collected, and an antibody subtype analysis was performed on the supernatant, and the binding activity on C5aR positive cells was determined by using FACS (the methods for detecting the binding activity are shown in examples 3A and 3B respectively).

According to the screening results of the 24-well plate, hybridoma cells with MFI value >50 in FACS experiment were selected as positive clones meeting the requirements. The hybridoma cells meeting the requirements were selected for subcloning in a 96-well plate by using limiting dilution, and were cultured in DMEM medium (purchased from Invitrogen) containing 10% (w/w) FBS under the conditions of 37° C. and 5% (v/v) CO₂. After 10 days of subcloning, preliminary screening was performed by using Acumen, and a single positive monoclone was selected and amplified into a 24-well plate for further culture. 3 days later, the positive antigen binding was determined by using FACS, and the biological activity was evaluated by using C5aR receptor-ligand binding experiment (the evaluation standard was MFI value >50 in FACS experiment).

According to the test results of the sample in the 24-well plate, the best clone was selected, and the best clone was amplified and cultured in DMEM medium (purchased from Invitrogen) containing 10% (w/w) FBS under the conditions of 37° C. and 5% (v/v) CO₂, then was cryopreserved in liquid nitrogen, and then the hybridoma cells of the present invention were obtained and can be used for subsequent antibody production and purification.

### Example 2

### Production and Purification of Lead Antibody

The antibody produced by the hybridoma cells had a low antibody concentration of only about 1-10 μg/milliliter, and the concentration varied greatly. Furthermore, multiple proteins in the medium produced by cell culture and the fetal bovine serum ingredient contained in the medium interfered with many biological activity analysis methods in varying degrees, and therefore, it was necessary to perform a small-scale (1-5 milligrams) antibody production and purification.

The hybridoma cells obtained in example 1 were inoculated into a T-75 cell culture flask and were acclimated and passaged for 3 generations by using a production medium (Hybridoma serum free medium, purchased from Invitrogen). When the cells had a good growing status, they were inoculated into a roller bottle for tissue culture. 500 milliliter production medium was added into each 2 litre roller bottle for tissue culture, and the inoculated cell density was 1.0×10⁵ cells/milliliter. The bottle cap was screwed down, and the roller bottle was placed onto a rotary machine in a 37° C. incubator, with the rotate speed being 3 revolutions/minute. After a continuous rotating culture of 14 days, the cell culture fluid was collected, the cells were removed by filtration, and the culture supernatant was filtered with a 0.45 micron filter membrane until it was clear. The clear culture supernatant can be purified immediately or cryopreserved at −30° C.

Monoclonal antibodies in the clear culture supernatant of the hybridoma cells (300 mL) was purified by using a 2 mL protein G column (purchased from GE Healthcare). First, the protein G column was balanced with an equilibration buffer (PBS phosphate buffer, pH 7.2), then the clear culture supernatant was loaded onto the protein G column, and the flow rate was controlled at 3 mL/minute. After the loading finished, the protein G column was washed with the equilibration buffer at a volume of 4 column bed volumes of the protein G column. Anti-C5aR antibody binding on the protein G column was eluted with an eluent (0.1 M glycinate buffer, pH 2.5), and the elution condition was monitored by an ultraviolet detector (A280 ultraviolet absorption peak). The eluted antibody was collected, into which added 10% 1.0 M Tris-HCl buffer for neutralizing the pH, wherein the percentage is the volume percentage, then was dialyzed with PBS phosphate buffer overnight immediately. The next day, the dialysate was changed once and was further dialyzed for 3 hours. The dialyzed anti-C5aR antibody was collected, was subjected to a sterile filtration with a 0.22 micron filter and stored aseptically, then the purified anti-C5aR antibody was obtained.

The purified C5aR antibody was detected and analyzed for protein concentration (A280/1.4), purity and endotoxin (Lonza kit) etc. The results are shown in table 6, and it is found that the endotoxin concentration of the final antibody product was within 1.0 EU/milligram, so that a purified anti-C5aR antibody was obtained.

TABLE 6
Detection and analysis of purified anti-C5aR antibody

| | | Protein concentration | Endotoxin |
|---|---|---|---|
| Clone numbers | Antibody purity | (milligram/milliliter) | (EU/milligram) |
| 5F8E2C11 | >90% | 2.06 | 0.21 |
| 42B5G7D1 | >90% | 0.27 | 0.57 |
| 43E8F5B6 | >90% | 0.16 | 0.14 |
| 46H2A11C7 | >90% | 1.17 | 0.22 |
| 2A12B2B2 | >90% | 0.82 | 0.44 |
| 8B5D1A9 | >90% | 0.36 | 0.39 |
| 9D5A12G7 | >90% | 0.91 | 0.22 |

### Example 3

### Detection and Identification of Lead Antibody

A. Binding of antibody and C5aR protein was detected by enzyme linked immunosorbent assay (ELISA) The purified anti-C5aR antibody obtained in example 2 was cross-reacted with 5 polypeptides of extracellular domains of C5aR, i.e. the purified immunogen A obtained in example 1, respectively.

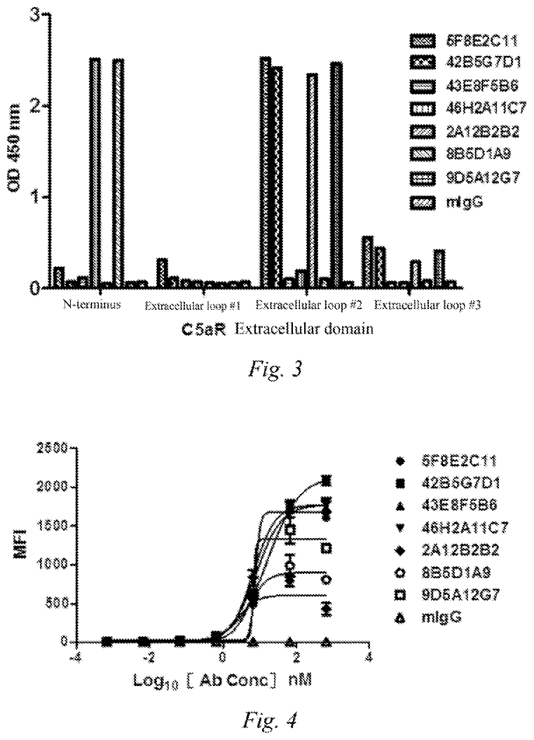

The 5 polypeptides of the purified immunogen A obtained in example 1 were diluted to a final concentration of 1.0 μg/mL with PBS respectively, then were added into a 96-well ELISA plate at 100 μl/well. The plate was sealed with a plastic film and incubated at 4° C. overnight. The next day, the plate was washed twice with a plate washing solution [PBS containing 0.01% (v/v) Tween 20], and a blocking solution [PBS containing 0.01% (v/v) Tween 20 and 1% (v/v) BSA] was added for blocking 2 hours at room temperature. The blocking solution was discarded, and the purified anti-C5aR antibody obtained in example 2 was added at 100 μl/well. After incubating at 37° C. for 2 hours, the plate was washed 3 times with the plate washing solution [PBS containing 0.01% (v/v) Tween 20]. A HRP (horseradish peroxidase) labelled second antibody (purchased from Sigma) was added, after incubating at 37° C. for 2 hours, the plate was washed 3 times with the plate washing solution [PBS containing 0.01% (v/v) Tween 20]. TMB substrate was added at 100 μl/well, after incubating at room temperature for 30 minutes, a stop solution (1.0 N HCl) was added at 100 μl/well. A450 nm value was read by using an ELISA plate reader (SpectraMax 384plus, purchased from Molecular Device), and the results are shown in FIG. 3 and table 7. Table 7 shows that the binding site of the purified antibody and C5aR are mainly at the N-terminus and the second extracellular loop (ECL #2). In the experiment, the IgG control was human IgG, and the data in the table was OD_{450 nm} value.

TABLE 7
Detection of the binding of anti-C5aR antibody
and specific epitopes of C5aR via ELISA

| | Extracellular domain of C5aR | | | |
|---|---|---|---|---|
| OD_{450 nm} | Extracellular | Extracellular | Extracellular | Extracellular |
| Clone numbers | N-terminus | loop-1 | loop-2 | loop-3 |
| 5F8E2C11 | 0.22 | 0.31 | 2.52 | 0.56 |
| 42B5G7D1 | 0.07 | 0.11 | 2.41 | 0.44 |
| 43E8F5B6 | 0.12 | 0.08 | 0.1 | 0.06 |
| 46H2A11C7 | 2.51 | 0.07 | 0.19 | 0.06 |
| 2A12B2B2 | 0.05 | 0.06 | 2.34 | 0.29 |
| 8B5D1A9 | 2.5 | 0.05 | 0.1 | 0.08 |
| 9D5A12G7 | 0.06 | 0.06 | 2.46 | 0.41 |
| IgG control | 0.07 | 0.07 | 0.06 | 0.07 |

B. Binding of antibody and C5aR expressing cell was detected by fluorescence activated cell sorting (FACS)

pIRES plasmid containing the nucleotide sequence encoding the full-length amino acid sequence of human C5aR of step (II) in example 1 was introduced into CHOK1 cell line, a stable CHOK1 cell line containing human C5aR (herein referred to as stable CHOK1-hC5aR cell line) was obtained, then amplified and cultured in a T-75 cell culture flask to 90% confluence, the medium was sucked out, the cells were washed twice with PBS buffer (phosphate buffer saline, purchased from Invitrogen), then were treated with an enzyme-free cell dissociation fluid (Versene solution, purchased from Invitrogen), and the cells were collected. The cells were washed twice with PBS buffer, and were counted, then were diluted to 2×10⁶ cells per milliliter with PBS buffer, and 2% calf serum blocking solution was added, wherein the percentage is the mass percentage. The cells were incubated at room temperature for 15 minutes, then were washed twice with PBS buffer by centrifugation. The collected cells were suspended to 3×10⁶ cells/mL with FACS buffer (PBS containing 2% FBS, wherein the percentage is the mass percentage). The cells were added into a 96-well FACS reaction plate at 100 microliters/well, the test sample the purified anti-C5aR antibody obtained in example 2 was added at 100 microliters/well, and then the plate was incubated at 4° C. for 1 hour. The cells were washed twice with FACS buffer by centrifugation, a fluorescence (Alexa 488) labelled secondary antibody (purchased from Invitrogen) was add at 100 microliters/well, and then the plate was incubated at 4° C. for 1 hour. The cells were washed 3 times with FACS buffer by centrifugation, a fixing solution [4% (v/v) paraformaldehyde] was added at 100 microliters/well for suspending the cells, 10 minutes later, the cells were washed twice with FACS buffer by centrifugation. The cells were suspended in 100 microliter of FACS buffer, then the results were detected and analyzed by using FACS (FACS Calibur, purchased from BD). The results are shown in FIG. 4 and table 8. Table 8 shows that the antibody to be detected can bind C5aR on the cell surface. In the experiment, the mIgG control was murine IgG, and was used as a negative control. The data in table 8 is the mean fluorescence intensity value of the cell population measured by MFI.

TABLE 8
Detection of the binding reaction between anti-C5aR antibody and CHOK1-hC5aR via FACS

| Antibody | | | | | | | | |
|---|---|---|---|---|---|---|---|---|
| concentration | | | | | | | | |
| (nM) | 5F8E2C11 | | 42B5G7D1 | | 43E8F5B6 | | 46H2A11C7 | |
| 666.0000 | 1570.47 | 1638.13 | 2141.84 | 2024.41 | 1671.53 | 1815.11 | 1669.01 | 1855.59 |
| 66.6000 | 1676.66 | 1835.2 | 1725.06 | 1691.91 | 1673.04 | 1803.63 | 1510.44 | 1811.06 |
| 6.6600 | 508.96 | 471.69 | 642.2 | 570.22 | 935.96 | 827.55 | 838.72 | 733.57 |
| 0.6660 | 52.75 | 50.3 | 78.1 | 70.7 | 102.8 | 93.48 | 89.38 | 82.28 |
| 0.0666 | 10.37 | 9.53 | 13.69 | 12.23 | 17.71 | 16.29 | 15.41 | 14.08 |
| 0.0067 | 3.67 | 2.89 | 4.28 | 3.43 | 4.69 | 3.86 | 4.33 | 3.45 |
| 0.0007 | 2.85 | 2.1 | 3.01 | 2.22 | 2.91 | 2.08 | 2.71 | 2.13 |
| 0.0000 | 2.69 | 2 | 2.78 | 2.01 | 2.74 | 2.05 | 2.64 | 1.97 |
| Antibody | | | | | | | | |
| concentration | | | | | | | | |
| (nM) | 2A12B2B2 | | 8B5D1A9 | | 9D5A12G7 | | mIgG | |
| 666.0000 | 357.94 | 506.97 | 775.94 | 839.68 | 1234.64 | 1187.11 | 3.22 | 2.21 |
| 66.6000 | 721.56 | 855.38 | 841.31 | 1130.71 | 1271.99 | 1612.69 | 3.18 | 2.14 |
| 6.6600 | 520.14 | 484.06 | 591.11 | 526.25 | 647.7 | 573.37 | 2.88 | 2.01 |
| 0.6660 | 76.77 | 69.38 | 78.96 | 71.97 | 77.92 | 74.37 | 2.96 | 1.99 |
| 0.0666 | 13.97 | 12.35 | 13.52 | 12.19 | 13.57 | 12.8 | 2.81 | 2 |
| 0.0067 | 4.17 | 3.29 | 3.97 | 3.17 | 3.93 | 3.25 | 2.79 | 2.01 |
| 0.0007 | 2.92 | 2.17 | 2.82 | 2.06 | 2.8 | 2.12 | 2.78 | 2.03 |
| 0.0000 | 2.87 | 2.08 | 2.81 | 2.06 | 2.65 | 2.01 | 2.69 | 1.99 |

### Example 4

Downstream signaling pathway mediated by C5a-C5aR was blocked by anti-C5aR antibody, which was detected by FLIPR calcium detection experiment

FLIPR® calcium assay evaluation kit (Product #R8172) was purchased from Molecular Devices, and the specific experimental steps followed the instructions of the kit.

The stable CHOK1-hC5aR cell line obtained in step (II) of example 1 was further engineered, into which Ga15 protein was introduced (wherein the accession number of Ga15 protein in NCBI database was NM 002068; the transfection used X-treme GENE HP DNA Transfection Reagent, purchased from Roche, Cat #06366236001, and operating according to the instructions), the stable CHOK1-Ga15-hC5aR cell line was obtained by antibiotic screening.

The CHOK1-Ga15-hC5aR cells were amplified and cultured in a T-75 cell culture flask to 90% confluence, the medium was sucked out, the cells were washed twice with PBS buffer, then were treated with an enzyme-free cell dissociation fluid (Versene solution, purchased from Life technology), and the cells were collected. The cells were counted, then were diluted to 2×10⁵ cells per milliliter with F-12K medium containing 10% (w/w) calf serum. The cells were added into a 384-well cell plate at 50 microliter/well, and the plate was placed in a incubator with 5% CO₂ and at 37° C. for culture overnight. The next day, the 384-well plate was taken out, the medium was discarded, 40 microliter of a fluorescent dye (purchased from FLIPR® Calcium Assay Evaluation Kit, Product #R8172) was added, then was left in the dark at room temperature for 1 hour. The purified anti-C5aR antibody obtained in example 2 was diluted to 150 μg/ml with a buffer [HBSS buffer containing 20 mM HEPES and 0.1% (w/w) BSA], then an antibody dilution solution was obtained. The antibody dilution solution was added into the 384-well plate at 10 microliter/well, then was left in the dark at room temperature for 1 hour. A 384-well plate and the 384-well cell plate with 30 microliter C5a solution diluted to 1 nM in each well were placed into a FLIPR instrument for detection. The results are shown in FIG. 5 and table 9. Table 9 shows that the antibody to be detected can bind C5aR on the cell surface, thereby blocking the calcium flow signal mediated by C5a. In the experiment, the mIgG control was murine IgG, and the data in the table is the mean fluorescence intensity value of the cell population measured by MFI.

TABLE 9
Detection of the blocking of C5a-mediated calcium flow signal by anti-C5aR antibody via FLIPR

| Antibody | | | | | | | | |
|---|---|---|---|---|---|---|---|---|
| concentration | | | | | | | | |
| (μg/mL) | 5F8E2C11 | | 42B5G7D1 | | 43E8F5B6 | | 46H2A11C7 | |
| 30.0 | 827.03 | 863.35 | 485.27 | 454.27 | 446.23 | 338.62 | 1085.38 | 1191.18 |
| 10.0 | 797.24 | 895.94 | 681.07 | 718.11 | 397.23 | 507.98 | 879.35 | 1075.44 |
| 3.3 | 1374.03 | 1556.11 | 1278.64 | 1170.45 | 295.80 | 433.19 | 923.29 | 817.95 |
| 1.1 | 1806.23 | 1794.01 | 1760.48 | 1749.46 | 808.17 | 589.97 | 1384.61 | 1339.30 |
| 0.4 | 1967.10 | 1752.24 | 2000.48 | 1745.87 | 1372.22 | 1312.72 | 1824.36 | 1682.61 |
| 0.1 | 1711.82 | 1637.65 | 1625.75 | 1692.17 | 2021.94 | 1910.79 | 1845.73 | 1729.32 |
| 0.01 | 1923.17 | 2046.19 | 2107.57 | 1756.84 | 2012.26 | 1833.66 | 1910.14 | 1889.47 |
| 0.0 | 1976.06 | 1791.64 | 1970.25 | 1824.82 | 2001.02 | 1907.01 | 2008.07 | 1807.04 |
| Antibody | | | | | | | | |
| concentration | | | | | | | | |
| (μg/mL) | 2A12B2B2 | | 8B5D1A9 | | 9D5A12G7 | | mIgG | |
| 30.0 | 322.34 | 471.69 | 1369.23 | 1683.06 | 992.52 | 1394.59 | 1588.93 | 1590.11 |
| 10.0 | 720.03 | 595.47 | 1715.78 | 1781.61 | 1200.64 | 1231.27 | 1725.72 | 1854.17 |
| 3.3 | 934.79 | 1092.10 | 1770.90 | 1791.11 | 1644.81 | 1493.31 | 1869.76 | 1835.18 |
| 1.1 | 1470.57 | 1197.07 | 1591.06 | 1713.85 | 1534.88 | 1653.67 | 1830.42 | 1789.36 |
| 0.4 | 1524.80 | 1615.58 | 1739.48 | 1923.29 | 1626.98 | 1637.51 | 1920.60 | 1607.72 |
| 0.1 | 1692.38 | 1540.62 | 1651.81 | 1734.71 | 1525.84 | 1592.57 | 2077.51 | 1743.77 |
| 0.01 | 1707.58 | 1423.92 | 1764.89 | 1727.93 | 1497.71 | 1712.67 | 2111.26 | 1885.36 |
| 0.0 | 1780.19 | 1604.75 | 1549.45 | 1840.01 | 1654.63 | 1537.88 | 1865.55 | 1688.43 |

### Example 5

Detection of the blocking of C5a-induced cell migration by anti-C5aR antibody via human neutrophil chemotaxis experiment

### (I) Obtaining Neutrophils PMNs in Peripheral Blood by Separating the Whole Blood Using Ficoll

Fresh whole blood was diluted with phosphate buffer PBS at 1:1 volume ratio to obtain a diluted whole blood. The diluted whole blood was gently spread on Ficoll liquid surface (purchased from GE Healthcare) by using a sterile pipette, with the volume ratio of Ficoll and the diluted whole blood being 3:4, and shaking and mixing were avoided. The liquid was subjected to a gradient centrifugation at 400 g rotate speed and at room temperature 20° C. for 30 minutes. In the centrifuged centrifuge tube, there was four divided layers: the top layer was plasma, the middle white film layer was mononuclear lymphocytes, and the two layers below were Ficoll and red blood cell-neutrophil layers respectively. The plasma, white film layer and Ficoll were discarded, precipitates of the red blood cell-neutrophil layer were retained, which was diluted to 25 mL with PBS, then 25 mL 3% (w/w) Dextran (molecular weight of 500,000 and purchased from Shanghai yuanye Bio-Technology Co., Ltd) was added. The tube was standing at room temperature for 20 minutes, then the red blood cell sedimentation was performed. The supernatant was taken, then was centrifuged at 500 g rotate speed and at room temperature for 10 minutes, then the supernatant was discarded, and the cell pellet was taken. The cell pellet was resuspended with sterile water to 25 mL, the centrifuge tube was turned upside down for adequately mixing. After being reacted for 28 seconds, the equal volume of 1.8% (w/w) sodium chloride solution (purchased from Sigma) was added to stop the red blood cell lysis. The tube was centrifuged at room temperature and at 500 g rotate speed for 5 minutes, and the supernatant was discarded. The cell pellet was resuspended with a chemotactic buffer (a mixed liquid of 49% RPMI1640, 49% M199 and 2% dialyzed FBS, wherein the percentage is the mass percentage) to 25 mL. The cells were counted and centrifuged. Finally, the neutrophils were resuspended in the chemotactic buffer, and the cell concentration was adjusted to 2×10⁷ cells per milliliter, then the human peripheral blood neutrophils were obtained.

### (II) Chemotaxis Experiment of Human Peripheral Blood Neutrophils

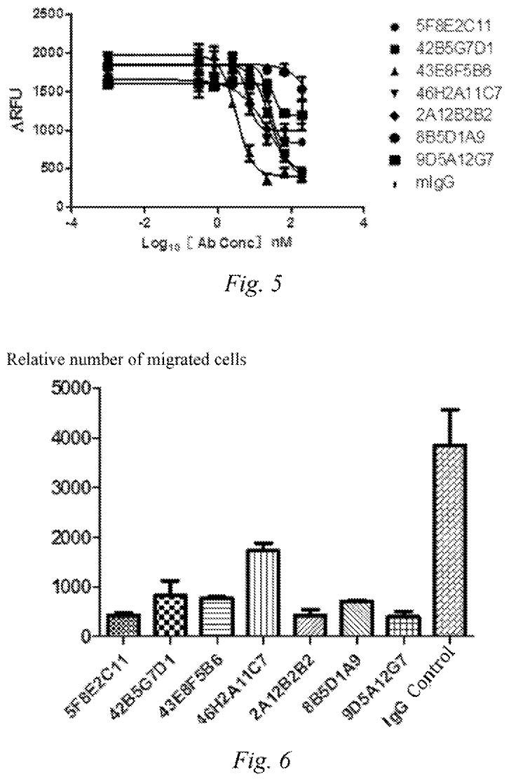

The human peripheral blood neutrophils obtained in step (I) of example 5 were spread onto a 96-well cell culture plate at 2×10⁶ cells/100 microliter/well. The anti-C5aR antibody in the concentration range from 0.5 μg/mL to 10 φg/mL [the purified anti-C5aR antibody obtained in example 2 was diluted with a chemotactic buffer containing 49% (v/v) RPMI1640, 49% (v/v) M199 and 0.02% (v/v) BSA] was added into the 96-well cell culture plate, and the plate was incubated in a 37° C. and 5% (v/v) CO₂ incubator for 20 minutes. The incubated cells were added into the upper chamber of a 24-well Transwell with 3.0 μm well diameter (purchased from Corning), then the upper chamber of the Transwell was placed into the lower chamber containing 10 nM C5a protein, and the Transwell was incubated in a 37° C. and 5% (v/v) CO₂ incubator for 30 minutes, during the incubating, some cells migrated into the lower chamber. The cells migrated into the lower chamber were transferred into another 96-well plate, then 100 μL Celltiter-Glo (purchased from Promega) was added for measurement. The results are shown in FIG. 6 and table 10. The data of FIG. 6 shows that the antibody to be detected had the activity of inhibiting the migration of human neutrophils. Table 10 shows that the antibody to be detected can block the cell migration induced by C5a. In the experiment, the IgG control was murine IgG, and the data in the table was the mean fluorescence intensity value measured by Celltiter-Glo.

TABLE 10
Detection of the blocking of C5a-induced cell migration
by anti-C5aR antibody via human neutrophil chemotaxis experiment

| Fluorescence | | | | |
|---|---|---|---|---|
| intensity value | 5F8E2C11 | 42B5G7D1 | 43E8F5B6 | 46H2A11C7 |
| Parallel sample 1 | 574.97 | 1200.89 | 844.26 | 1455.62 |
| Parallel sample 2 | 429.41 | 1477.46 | 807.87 | 1506.57 |
| Parallel sample 3 | 400.30 | 298.40 | 705.98 | 1928.70 |
| Parallel sample 4 | 312.96 | 334.79 | 756.92 | 2037.87 |
| Fluorescence | | | | |
| intensity value | 2A12B2B2 | 8B5D1A9 | 9D5A12G7 | IgG control |
| Parallel sample 1 | 669.59 | 655.03 | 174.68 | 5174.74 |
| Parallel sample 2 | 596.81 | 713.26 | 211.07 | 5000.06 |
| Parallel sample 3 | 232.90 | 705.98 | 574.97 | 2576.45 |
| Parallel sample 4 | 203.79 | 749.65 | 611.36 | 2641.96 |

### Example 6

Determination of amino acid sequences of light and heavy chain variable regions Total RNA separation: After the supernatant obtained by the subclone culture in example 1 was detected for antigen binding (that is, was subjected to the detection, identification and activity assay of examples 3-5), 5×10⁷ hybridoma cells were collected by centrifugation, 1 mL Trizol was added, and the mixture was mixed homogeneously and transferred into a 1.5 mL centrifuge tube. The tube was standing at room temperature for 5 minutes. 0.2 mL chloroform was added. The tube was shaken for 15 seconds, then was standing for 2 minutes, and was centrifuged at 4° C. and at 12000 g for 5 minutes, then the supernatant was transferred into a new 1.5 mL centrifuge tube. 0.5 mL isopropanol was added, the liquid in the tube was mixed homogeneously and gently, then the tube was standing at room temperature for 10 minutes, was centrifuged at 4° C. and at 12000 g for 15 minutes, then the supernatant was discarded. 1 mL 75% ethanol (wherein the percentage is the volume percentage) was added, the precipitate was washed gently, then the tube was centrifuged at 4° C. and at 12000 g for 5 minutes, the supernatant was discarded, the precipitate was dried in the air, then DEPC treated H₂O was added for dissolution (in 55° C. water bath for 10 minutes to promote the dissolution), then the total RNA was obtained.

Reverse transcription and PCR: 1 μg total RNA was taken to formulate a 20 μL system, into which a reverse transcriptase was added, then was reacted at 42° C. for 60 minutes, and was reacted at 7° C. for 10 minutes, then the reaction was terminated. A 50 μL PCR system was formulated. The system comprised 1 μL cDNA, 25 pmol of each primer, 1 μL DNApolymerase, a corresponding buffer system and 250 μmol dNTPs. The PCR program was set: pre-denaturation at 95° C. for 3 minutes, denaturation at 95° C. for 30 seconds, annealing at 55° C. for 30 seconds, extension at 72° C. for 35 seconds, 35 cycles, additionally, extension at 72° C. for 5 minutes, then the PCR product was obtained. The kit used for reverse transcription was PrimeScript RT Master Mix, purchased from Takara, Cat #RR036. The reagent used for PCR was Q5 high-fidelity DNA polymerase, purchased from NEB, Cat #M0492.

Cloning and sequencing: 5 μL PCR product was taken for an agarose gel electrophoresis detection, and the positive detection sample was purified by using a column recovery kit, wherein the recovery kit was NucleoSpin® Gel & PCR Clean-up, purchased from MACHEREY-NAGEL, Cat #740609. Ligation reaction: 50 ng sample, 50 ng T vector, 0.5 μL ligase, 1 μL buffer, and 10 μL reaction system, were reacted at 16° C. for half an hour to obtain a ligation product, wherein the ligation reagent was T4 DNA ligase, purchased from NEB, Cat #M0402; 5 μL ligation product was taken and added into 100 μL competent cells (Ecos 101 competent cells, purchased from Yeastern, Cat #FYE607), ice bath for 5 minutes. Then the cells were heat shocked in 42° C. water bath for 1 minute, was placed back onto ice for 1 minute, and 650 μL of an antibiotic-free SOC medium was added, then recovered on a 37° C. shaker at a speed of 200 RPM for 30 minutes, and 200 μL was taken out and spread onto LB solid medium containing antibiotics, then was cultured in a 37° C. incubator overnight. The next day, primers M13F and M13R on T vector were used for formulating a 30 μL PCR system. A colony PCR was performed, wherein a colony was dipped into the PCR system by a pipette tip, then was blown and sucked, and 0.5 μL was sucked out and placed onto another LB solid culture dish containing 100 nM ampicillin for strain preservation. After the PCR reaction was completed, 5 μL was taken out for an agarose gel electrophoresis detection, and the positive sample was sequenced. For the sequencing steps, see Kabat, Sequences of Proteins of Immunological Interest, National Institutes of Health, Bethesda, Md. (1991).

The sequencing results are shown in tables 11 and 12:

TABLE 11
Numbers of protein sequence of anti-C5aR antibody

| | Heavy chain protein | | | | Light chain protein | | | |
|---|---|---|---|---|---|---|---|---|
| Clone | Variable | CD | CD | CD | Variable | CD | CD | CD |
| numbers | region | R1 | R2 | R3 | region | R1 | R2 | R3 |
| 5F8E2C11 | 1 | 2 | 3 | 4 | 5 | 6 | 7 | 8 |
| 42B5G7D1 | 9 | 10 | 11 | 12 | 13 | 14 | 15 | 16 |
| 43E8F5B6 | 17 | 18 | 19 | 20 | 21 | 22 | 23 | 24 |
| 46H2A11C7 | 25 | 26 | 27 | 28 | 29 | 30 | 31 | 32 |
| 2A12B2B2 | 33 | 34 | 35 | 36 | 37 | 38 | 39 | 40 |
| 8B5D1A9 | 41 | 42 | 43 | 44 | 45 | 46 | 47 | 48 |
| 9D5A12G7 | 49 | 50 | 51 | 52 | 53 | 54 | 55 | 56 |

The numbers in table 11 are the numbers “SEQ ID Nos.” in the sequence listing, for example, the amino acid sequence of the heavy chain protein variable region of 5F8E2C11 is SEQ ID No. 1 in the sequence listing, and the amino acid sequence of CDR1 domain in the heavy chain protein variable region of 5F8E2C11 is SEQ ID No. 2 in the sequence listing.

TABLE 12
Numbers of gene sequence of anti-C5aR antibody

| | Heavy chain proteins | Light chain proteins |
|---|---|---|
| Clone numbers | variable region | Variable region |
| 5F8E2C11 | 57 | 58 |
| 42B5G7D1 | 59 | 60 |
| 43E8F5B6 | 61 | 62 |
| 46H2A11C7 | 63 | 64 |
| 2A12B2B2 | 65 | 66 |
| 8B5D1A9 | 67 | 68 |
| 9D5A12G7 | 69 | 70 |

The numbers in table 12 are the numbers “SEQ ID Nos.” in the sequence listing, for example, the nucleotide sequence encoding the heavy chain protein variable region of 5F8E2C11 is SEQ ID No. 33 in the sequence listing.

The nucleotide sequence encoding CDR1 in the heavy chain protein variable region of 5F8E2C11 is positions 76 to 105 of SEQ ID No. 57 in the sequence listing;

the nucleotide sequence encoding CDR2 in the heavy chain protein variable region of 5F8E2C11 is positions 148 to 195 of SEQ ID No. 57 in the sequence listing;

the nucleotide sequence encoding CDR3 in the heavy chain protein variable region of 5F8E2C11 is positions 292 to 321 of SEQ ID No. 57 in the sequence listing;

the nucleotide sequence encoding CDR1 in the light chain protein variable region of 5F8E2C11 is positions 70 to 120 of SEQ ID No. 58 in the sequence listing;

the nucleotide sequence encoding CDR2 in the light chain protein variable region of 5F8E2C11 is positions 166 to 186 of SEQ ID No. 58 in the sequence listing;

the nucleotide sequence encoding CDR3 in the light chain protein variable region of 5F8E2C11 is positions 283 to 306 of SEQ ID No. 58 in the sequence listing;

the nucleotide sequence encoding CDR1 in the heavy chain protein variable region of 42B5G7D1 is positions 76 to 105 of SEQ ID No. 59 in the sequence listing;

the nucleotide sequence encoding CDR2 in the heavy chain protein variable region of 42B5G7D1 is positions 148 to 198 of SEQ ID No. 59 in the sequence listing;

the nucleotide sequence encoding CDR3 in the heavy chain protein variable region of 42B5G7D1 is positions 295 to 336 of SEQ ID No. 59 in the sequence listing;

the nucleotide sequence encoding CDR1 in the light chain protein variable region of 42B5G7D1 is positions 70 to 117 of SEQ ID No. 60 in the sequence listing;

the nucleotide sequence encoding CDR2 in the light chain protein variable region of 42B5G7D1 is positions 163 to 183 of SEQ ID No. 60 in the sequence listing;

the nucleotide sequence encoding CDR3 in the light chain protein variable region of 42B5G7D1 is positions 280 to 306 of SEQ ID No. 60 in the sequence listing;

the nucleotide sequence encoding CDR1 in the heavy chain protein variable region of 43E8F5B6 is positions 76 to 105 of SEQ ID No. 61 in the sequence listing;

the nucleotide sequence encoding CDR2 in the heavy chain protein variable region of 43E8F5B6 is positions 148 to 198 of SEQ ID No. 61 in the sequence listing;

the nucleotide sequence encoding CDR3 in the heavy chain protein variable region of 43E8F5B6 is positions 295 to 339 of SEQ ID No. 61 in the sequence listing;

the nucleotide sequence encoding CDR1 in the light chain protein variable region of 43E8F5B6 is positions 70 to 105 of SEQ ID No. 62 in the sequence listing;

the nucleotide sequence encoding CDR2 in the light chain protein variable region of 43E8F5B6 is positions 151 to 171 of SEQ ID No. 62 in the sequence listing;

the nucleotide sequence encoding CDR3 in the light chain protein variable region of 43E8F5B6 is positions 268 to 294 of SEQ ID No. 62 in the sequence listing;

the nucleotide sequence encoding CDR1 in the heavy chain protein variable region of 46H2A11C7 is positions 76 to 105 of SEQ ID No. 63 in the sequence listing;

the nucleotide sequence encoding CDR2 in the heavy chain protein variable region of 46H2A11C7 is positions 148 to 198 of SEQ ID No. 63 in the sequence listing;

the nucleotide sequence encoding CDR3 in the heavy chain protein variable region of 46H2A11C7 is positions 295 to 312 of SEQ ID No. 63 in the sequence listing;

the nucleotide sequence encoding CDR1 in the light chain protein variable region of 46H2A11C7 is positions 70 to 117 of SEQ ID No. 64 in the sequence listing;

the nucleotide sequence encoding CDR2 in the light chain protein variable region of 46H2A11C7 is positions 163 to 183 of SEQ ID No. 64 in the sequence listing;

the nucleotide sequence encoding CDR3 in the light chain protein variable region of 46H2A11C7 is positions 280 to 306 of SEQ ID No. 64 in the sequence listing;

the nucleotide sequence encoding CDR1 in the heavy chain protein variable region of 2A12B2B2 is positions 76 to 105 of SEQ ID No. 65 in the sequence listing;

the nucleotide sequence encoding CDR2 in the heavy chain protein variable region of 2A12B2B2 is positions 148 to 198 of SEQ ID No. 65 in the sequence listing;

the nucleotide sequence encoding CDR3 in the heavy chain protein variable region of 2A12B2B2 is positions 295 to 303 of SEQ ID No. 65 in the sequence listing;

the nucleotide sequence encoding CDR1 in the light chain protein variable region of 2A12B2B2 is positions 70 to 102 of SEQ ID No. 66 in the sequence listing;

the nucleotide sequence encoding CDR2 in the light chain protein variable region of 2A12B2B2 is positions 148 to 168 of SEQ ID No. 66 in the sequence listing;

the nucleotide sequence encoding CDR3 in the light chain protein variable region of 2A12B2B2 is positions 265 to 291 of SEQ ID No. 66 in the sequence listing;

the nucleotide sequence encoding CDR1 in the heavy chain protein variable region of 8B5D1A9 is positions 76 to 105 of SEQ ID No. 67 in the sequence listing;

the nucleotide sequence encoding CDR2 in the heavy chain protein variable region of 8B5D1A9 is positions 148 to 198 of SEQ ID No. 67 in the sequence listing;

the nucleotide sequence encoding CDR3 in the heavy chain protein variable region of 8B5D1A9 is positions 295 to 324 of SEQ ID No. 67 in the sequence listing;

the nucleotide sequence encoding CDR1 in the light chain protein variable region of 8B5D1A9 is positions 70 to 120 of SEQ ID No. 68 in the sequence listing;

the nucleotide sequence encoding CDR2 in the light chain protein variable region of 8B5D1A9 is positions 166 to 189 of SEQ ID No. 68 in the sequence listing;

the nucleotide sequence encoding CDR3 in the light chain protein variable region of 8B5D1A9 is positions 286 to 312 of SEQ ID No. 68 in the sequence listing;

the nucleotide sequence encoding CDR1 in the heavy chain protein variable region of 9D5A12G7 is positions 76 to 105 of SEQ ID No. 69 in the sequence listing;

the nucleotide sequence encoding CDR2 in the heavy chain protein variable region of 9D5A12G7 is positions 148 to 195 of SEQ ID No. 69 in the sequence listing;

the nucleotide sequence encoding CDR3 in the heavy chain protein variable region of 9D5A12G7 is positions 292 to 321 of SEQ ID No. 69 in the sequence listing;

the nucleotide sequence encoding CDR1 in the light chain protein variable region of 9D5A12G7 is positions 70 to 120 of SEQ ID No. 70 in the sequence listing;

the nucleotide sequence encoding CDR2 in the light chain protein variable region of 9D5A12G7 is positions 166 to 186 of SEQ ID No. 70 in the sequence listing;

and the nucleotide sequence encoding CDR3 in the light chain protein variable region of 9D5A12G7 is positions 283 to 306 of SEQ ID No. 70 in the sequence listing.

It should be understood that after reading the above content of the invention, those skilled in the art can make various changes or modifications to the invention, and these equivalent forms also fall within the scope defined by the appended claims of this application.

1. An isolated protein comprising one or more of the followings: a heavy chain CDR1, heavy chain CDR2 or heavy chain CDR3 of an anti-C5aR antibody, and/or one or more of the followings: a light chain CDR1, light chain CDR2 or light chain CDR3 of the anti-C5aR antibody, wherein the amino acid sequence of the heavy chain CDR1 is as shown in SEQ ID No. 2, SEQ ID No. 10, SEQ ID No. 18, SEQ ID No. 26, SEQ ID No. 34, SEQ ID No. 42 or SEQ ID No. 50 in the sequence listing; the amino acid sequence of the heavy chain CDR2 is as shown in SEQ ID No. 3, SEQ ID No. 11, SEQ ID No. 19, SEQ ID No. 27, SEQ ID No. 35, SEQ ID No. 43 or SEQ ID No. 51 in the sequence listing; the amino acid sequence of the heavy chain CDR3 is as shown in SEQ ID No. 4, SEQ ID No. 12, SEQ ID No. 20, SEQ ID No. 28, SEQ ID No. 36, SEQ ID No. 44 or SEQ ID No. 52 in the sequence listing;
the amino acid sequence of the light chain CDR1 is as shown in SEQ ID No. 6, SEQ ID No. 14, SEQ ID No. 22, SEQ ID No. 30, SEQ ID No. 38, SEQ ID No. 46 or SEQ ID No. 54 in the sequence listing; the amino acid sequence of the light chain CDR2 is as shown in SEQ ID No. 7, SEQ ID No. 15, SEQ ID No. 23, SEQ ID No. 31, SEQ ID No. 39, SEQ ID No. 47 or SEQ ID No. 55 in the sequence listing; the amino acid sequence of the light chain CDR3 is as shown in SEQ ID No. 8, SEQ ID No. 16, SEQ ID No. 24, SEQ ID No. 32, SEQ ID No. 40, SEQ ID No. 48 or SEQ ID No. 56 in the sequence listing;
or the amino acid sequence of the heavy chain CDR1 is as shown in an amino acid sequence which has at least 80% sequence homology with the amino acid sequence shown in SEQ ID No. 2, SEQ ID No. 10, SEQ ID No. 18, SEQ ID No. 26, SEQ ID No. 34, SEQ ID No. 42 or SEQ ID No. 50 in the sequence listing; the amino acid sequence of the heavy chain CDR2 is as shown in an amino acid sequence which has at least 80% sequence homology with the amino acid sequence shown in SEQ ID No. 3, SEQ ID No. 11, SEQ ID No. 19, SEQ ID No. 27, SEQ ID No. 35, SEQ ID No. 43 or SEQ ID No. 51 in the sequence listing; the amino acid sequence of the heavy chain CDR3 is as shown in an amino acid sequence which has at least 80% sequence homology with the amino acid sequence shown in SEQ ID No. 4, SEQ ID No. 12, SEQ ID No. 20, SEQ ID No. 28, SEQ ID No. 36, SEQ ID No. 44 or SEQ ID No. 52 in the sequence listing;
the amino acid sequence of the light chain CDR1 is as shown in an amino acid sequence which has at least 80% sequence homology with the amino acid sequence shown in SEQ ID No. 6, SEQ ID No. 14, SEQ ID No. 22, SEQ ID No. 30, SEQ ID No. 38, SEQ ID No. 46 or SEQ ID No. 54 in the sequence listing; the amino acid sequence of the light chain CDR2 is as shown in an amino acid sequence which has at least 80% sequence homology with the amino acid sequence shown in SEQ ID No. 7, SEQ ID No. 15, SEQ ID No. 23, SEQ ID No. 31, SEQ ID No. 39, SEQ ID No. 47 or SEQ ID No. 55 in the sequence listing; and the amino acid sequence of the light chain CDR3 is as shown in an amino acid sequence which has at least 80% sequence homology with the amino acid sequence shown in SEQ ID No. 8, SEQ ID No. 16, SEQ ID No. 24, SEQ ID No. 32, SEQ ID No. 40, SEQ ID No. 48 or SEQ ID No. 56 in the sequence listing.

2. The protein as defined in claim 1, wherein the amino acid sequence of the heavy chain CDR1 is as shown in SEQ ID No. 2 in the sequence listing, the amino acid sequence of the heavy chain CDR2 is as shown in SEQ ID No. 3 in the sequence listing, and the amino acid sequence of the heavy chain CDR3 is as shown in SEQ ID No. 4 in the sequence listing;
the amino acid sequence of the heavy chain CDR1 is as shown in SEQ ID No. 10 in the sequence listing, the amino acid sequence of the heavy chain CDR2 is as shown in SEQ ID No. 11 in the sequence listing, and the amino acid sequence of the heavy chain CDR3 is as shown in SEQ ID No. 12 in the sequence listing;
the amino acid sequence of the heavy chain CDR1 is as shown in SEQ ID No. 18 in the sequence listing, the amino acid sequence of the heavy chain CDR2 is as shown in SEQ ID No. 19 in the sequence listing, and the amino acid sequence of the heavy chain CDR3 is as shown in SEQ ID No. 20 in the sequence listing;
the amino acid sequence of the heavy chain CDR1 is as shown in SEQ ID No. 26 in the sequence listing, the amino acid sequence of the heavy chain CDR2 is as shown in SEQ ID No. 27 in the sequence listing, and the amino acid sequence of the heavy chain CDR3 is as shown in SEQ ID No. 28 in the sequence listing;
the amino acid sequence of the heavy chain CDR1 is as shown in SEQ ID No. 34 in the sequence listing, the amino acid sequence of the heavy chain CDR2 is as shown in SEQ ID No. 35 in the sequence listing, and the amino acid sequence of the heavy chain CDR3 is as shown in SEQ ID No. 36 in the sequence listing;
the amino acid sequence of the heavy chain CDR1 is as shown in SEQ ID No. 42 in the sequence listing, the amino acid sequence of the heavy chain CDR2 is as shown in SEQ ID No. 43 in the sequence listing, and the amino acid sequence of the heavy chain CDR3 is as shown in SEQ ID No. 44 in the sequence listing;
the amino acid sequence of the heavy chain CDR1 is as shown in SEQ ID No. 50 in the sequence listing, the amino acid sequence of the heavy chain CDR2 is as shown in SEQ ID No. 51 in the sequence listing, and the amino acid sequence of the heavy chain CDR3 is as shown in SEQ ID No. 52 in the sequence listing;
the amino acid sequence of the light chain CDR1 is as shown in SEQ ID No. 6 in the sequence listing, the amino acid sequence of the light chain CDR2 is as shown in SEQ ID No. 7 in the sequence listing, and the amino acid sequence of the light chain CDR3 is as shown in SEQ ID No. 8 in the sequence listing;
the amino acid sequence of the light chain CDR1 is as shown in SEQ ID No. 14 in the sequence listing, the amino acid sequence of the light chain CDR2 is as shown in SEQ ID No. 15 in the sequence listing, and the amino acid sequence of the light chain CDR3 is as shown in SEQ ID No. 16 in the sequence listing;
the amino acid sequence of the light chain CDR1 is as shown in SEQ ID No. 22 in the sequence listing, the amino acid sequence of the light chain CDR2 is as shown in SEQ ID No. 23 in the sequence listing, and the amino acid sequence of the light chain CDR3 is as shown in SEQ ID No. 24 in the sequence listing;
the amino acid sequence of the light chain CDR1 is as shown in SEQ ID No. 30 in the sequence listing, the amino acid sequence of the light chain CDR2 is as shown in SEQ ID No. 31 in the sequence listing, and the amino acid sequence of the light chain CDR3 is as shown in SEQ ID No. 32 in the sequence listing;
the amino acid sequence of the light chain CDR1 is as shown in SEQ ID No. 38 in the sequence listing, the amino acid sequence of the light chain CDR2 is as shown in SEQ ID No. 39 in the sequence listing, and the amino acid sequence of the light chain CDR3 is as shown in SEQ ID No. 40 in the sequence listing;
the amino acid sequence of the light chain CDR1 is as shown in SEQ ID No. 46 in the sequence listing, the amino acid sequence of the light chain CDR2 is as shown in SEQ ID No. 47 in the sequence listing, and the amino acid sequence of the light chain CDR3 is as shown in SEQ ID No. 48 in the sequence listing;
or the amino acid sequence of the light chain CDR1 is as shown in SEQ ID No. 54 in the sequence listing, the amino acid sequence of the light chain CDR2 is as shown in SEQ ID No. 55 in the sequence listing, and the amino acid sequence of the light chain CDR3 is as shown in SEQ ID No. 56 in the sequence listing.

3. An isolated protein comprising a heavy chain variable region of an anti-C5aR antibody and/or a light chain variable region of an anti-C5aR antibody, wherein the amino acid sequence of the heavy chain variable region is as shown in SEQ ID No. 1, SEQ ID No. 9, SEQ ID No. 17, SEQ ID No. 25, SEQ ID No. 33, SEQ ID No. 41 or SEQ ID No. 49 in the sequence listing; and the amino acid sequence of the light chain variable region is as shown in SEQ ID No. 5, SEQ ID No. 13, SEQ ID No. 21, SEQ ID No. 29, SEQ ID No. 37, SEQ ID No. 45 or SEQ ID No. 53 in the sequence listing.

4. The protein as defined in claim 3, wherein the amino acid sequence of the heavy chain variable region is as shown in SEQ ID No. 1 in the sequence listing, and the amino acid sequence of the light chain variable region is as shown in SEQ ID No. 5 in the sequence listing;
the amino acid sequence of the heavy chain variable region is as shown in SEQ ID No. 9 in the sequence listing, and the amino acid sequence of the light chain variable region is as shown in SEQ ID No. 13 in the sequence listing;
the amino acid sequence of the heavy chain variable region is as shown in SEQ ID No. 17 in the sequence listing, and the amino acid sequence of the light chain variable region is as shown in SEQ ID No. 21 in the sequence listing;
the amino acid sequence of the heavy chain variable region is as shown in SEQ ID No. 25 in the sequence listing, and the amino acid sequence of the light chain variable region is as shown in SEQ ID No. 29 in the sequence listing;
the amino acid sequence of the heavy chain variable region is as shown in SEQ ID No. 33 in the sequence listing, and the amino acid sequence of the light chain variable region is as shown in SEQ ID No. 37 in the sequence listing;
the amino acid sequence of the heavy chain variable region is as shown in SEQ ID No. 41 in the sequence listing, and the amino acid sequence of the light chain variable region is as shown in SEQ ID No. 45 in the sequence listing;
or the amino acid sequence of the heavy chain variable region is as shown in SEQ ID No. 49 in the sequence listing, and the amino acid sequence of the light chain variable region is as shown in SEQ ID No. 53 in the sequence listing.

5. The protein as defined in claim 1, wherein the protein further comprises an antibody heavy chain constant region and/or antibody light chain constant region.

6. The protein as defined in claim 5, wherein the antibody heavy chain constant region is a mouse antibody heavy chain constant region; and the antibody light chain constant region is a mouse antibody light chain constant region.

7. The protein as defined in claim 1, wherein the protein is a monoclonal antibody, full-length antibody protein, antigen-antibody binding domain protein fragment, bispecific antibody, polyspecific antibody, single chain antibody fragment, single domain antibody or single-domain antibody of the anti-C5aR antibody.

8. A nucleic acid which encodes the protein as defined in claim 1.

9. The nucleic acid as defined in claim 8, wherein the nucleic acid encoding the heavy chain variable region is as shown in SEQ ID No. 57 in the sequence listing, SEQ ID No. 59 in the sequence listing, SEQ ID No. 61 in the sequence listing, SEQ ID No. 63 in the sequence listing, SEQ ID No. 65 in the sequence listing, SEQ ID No. 67 in the sequence listing or SEQ ID No. 69 in the sequence listing; and/or the nucleotide sequence of the nucleic acid encoding the light chain variable region is as shown in SEQ ID No. 58 in the sequence listing, SEQ ID No. 60 in the sequence listing, SEQ ID No. 62 in the sequence listing, SEQ ID No. 64 in the sequence listing, SEQ ID No. 66 in the sequence listing, SEQ ID No. 68 in the sequence listing or SEQ ID No. 70 in the sequence listing.

10. The nucleic acid as defined in claim 9, wherein the nucleic acid encoding the heavy chain variable region is as shown in SEQ ID No. 57 in the sequence listing, and the nucleic acid encoding the light chain variable region is as shown in SEQ ID No. 58 in the sequence listing;
the nucleic acid encoding the heavy chain variable region is as shown in SEQ ID No. 59 in the sequence listing, and the nucleic acid encoding the light chain variable region is as shown in SEQ ID No. 60 in the sequence listing;
the nucleic acid encoding the heavy chain variable region is as shown in SEQ ID No. 61 in the sequence listing, and the nucleic acid encoding the light chain variable region is as shown in SEQ ID No. 62 in the sequence listing;
the nucleic acid encoding the heavy chain variable region is as shown in SEQ ID No. 63 in the sequence listing, and the nucleic acid encoding the light chain variable region is as shown in SEQ ID No. 64 in the sequence listing;
the nucleic acid encoding the heavy chain variable region is as shown in SEQ ID No. 65 in the sequence listing, and the nucleic acid encoding the light chain variable region is as shown in SEQ ID No. 66 in the sequence listing;
the nucleic acid encoding the heavy chain variable region is as shown in SEQ ID No. 67 in the sequence listing, and the nucleic acid encoding the light chain variable region is as shown in SEQ ID No. 68 in the sequence listing;
or the nucleic acid encoding the heavy chain variable region is as shown in SEQ ID No. 69 in the sequence listing, and the nucleic acid encoding the light chain variable region is as shown in SEQ ID No. 70 in the sequence listing.

11. A recombinant expression vector comprising the nucleic acid as defined in claim 8.

12. A recombinant expression transformant comprising the recombinant expression vector as defined in claim 11.

13. A method for preparing an anti-C5aR antibody, comprising the following steps: culturing the recombinant expression transformant as defined in claim 12, and obtaining the anti-C5aR antibody from the culture.

14. A method for detecting a cell overexpressing C5aR protein, comprising the following steps: contacting the protein as defined in claim 1 with a sample to be detected in vitro, then detecting the binding of the protein and the sample to be detected.


SEQUENCE LISTING (72 sequences)

SEQ ID NO: 1 - protein, 118 aa, Homo sapiens
QVQLRQSGPGLVQPSQSLSITCTVSGFSLTSYGVHWVRQSPGKGLEWLGVIWSGGSTDYNAAFISRLSISKDYSKSQIFLTMNSLQTDDTAIYYCARNSRLGNCFDYWGQGTTLTVSS

SEQ ID NO: 2 - protein, 10 aa, Homo sapiens
GFSLTSYGVH

SEQ ID NO: 3 - protein, 16 aa, Homo sapiens
VIWSGGSTDYNAAFIS

SEQ ID NO: 4 - protein, 10 aa, Homo sapiens
NSRLGNCFDY

SEQ ID NO: 5 - protein, 112 aa, Homo sapiens
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLPAFGGGTKLEIK

SEQ ID NO: 6 - protein, 17 aa, Homo sapiens
KSSQSLLNSRTRKNYLA

SEQ ID NO: 7 - protein, 7 aa, Homo sapiens
WASTRES

SEQ ID NO: 8 - protein, 8 aa, Homo sapiens
KQSYNLPA

SEQ ID NO: 9 - protein, 123 aa, Homo sapiens
QVQLQQSGPELVKPGASVKISCKASGYVFSNSWMNWVKQRPGKGLEWIGRIDPGDGDTKYNGKFKGKATLTADKSSSTAYMQLSSLTSEDSAVYFCVRFYYGSNYVGYYFDYWGQGTTLTVSS

SEQ ID NO: 10 - protein, 10 aa, Homo sapiens
GYVFSNSWMN

SEQ ID NO: 11 - protein, 17 aa, Homo sapiens
RIDPGDGDTKYNGKFKG

SEQ ID NO: 12 - protein, 14 aa, Homo sapiens
FYYGSNYVGYYFDY

SEQ ID NO: 13 - protein, 112 aa, Homo sapiens
DVLMTQTPLSLPVSLGDQASISCRSSQSIVQSNGNTYLEWYLQKPGQSPKLLIYKVSNRFSGVPDRFSGSGSGTDFTLKISRVEAEDLGVYYCFQGSLVPPTFGGGTKLEIK

SEQ ID NO: 14 - protein, 16 aa, Homo sapiens
RSSQSIVQSNGNTYLE

SEQ ID NO: 15 - protein, 7 aa, Homo sapiens
KVSNRFS

SEQ ID NO: 16 - protein, 9 aa, Homo sapiens
FQGSLVPPT

SEQ ID NO: 17 - protein, 124 aa, Homo sapiens
EVQLQQSGPELVKPGASVKISCKASGYTFTDYYMSWMKQSHGKSLEWIGDINPNNGDNIYNQKFKGKATLTVDRSSSTAYMDLRSLTSEDSAVYYCARWYYYVSSFSYWYFDVWGTGTTVTVSS

SEQ ID NO: 18 - protein, 10 aa, Homo sapiens
GYTFTDYYMS

SEQ ID NO: 19 - protein, 17 aa, Homo sapiens
DINPNNGDNIYNQKFKG

SEQ ID NO: 20 - protein, 15 aa, Homo sapiens
WYYYVSSFSYWYFDV

SEQ ID NO: 21 - protein, 108 aa, Homo sapiens
EIVLTQSPTTMAASPGEKITITCSATSSINSNYLHWYQQKPGFSPKLLIYRTSNLASGVPARFSGSGSGTSYSLTIGTMEAEDVATYYCQQGRTIPRTFGSGTKLEIK

SEQ ID NO: 22 - protein, 12 aa, Homo sapiens
SATSSINSNYLH

SEQ ID NO: 23 - protein, 7 aa, Homo sapiens
RTSNLAS

SEQ ID NO: 24 - protein, 9 aa, Homo sapiens
QQGRTIPRT

SEQ ID NO: 25 - protein, 115 aa, Homo sapiens
QVQLKQSGAELVKPGASVKISCRASGYTFTDYFINWVKQRPGQGLEWIGKIGPGSGNIYQNEKFKDKATLTTDTSSSTAYMQLSSLTSEDSAVYFCARWGIQRGWGQGTTLTVSS

SEQ ID NO: 26 - protein, 10 aa, Homo sapiens
GYTFTDYFIN

SEQ ID NO: 27 - protein, 17 aa, Homo sapiens
KIGPGSGNIYQNEKFKD

SEQ ID NO: 28 - protein, 6 aa, Homo sapiens
WGIQRG

SEQ ID NO: 29 - protein, 112 aa, Homo sapiens
DVVMTQTPLTLSVTIGQPASISCKSSQSLLDSDGKTYLNWLLQRPGQSPKRLFFLVSKRDSEVPDRFTGSGSGTDFTLKISRVEAEDLGVYYCWQVTHFPYTFGGGTKLEIK

SEQ ID NO: 30 - protein, 16 aa, Homo sapiens
KSSQSLLDSDGKTYLN

SEQ ID NO: 31 - protein, 7 aa, Homo sapiens
LVSKRDS

SEQ ID NO: 32 - protein, 9 aa, Homo sapiens
WQVTHFPYT

SEQ ID NO: 33 - protein, 112 aa, Homo sapiens
QVQLQQPGADLVKPGASVKMSCKASGYILTTYWITWVKQRPGQGLEWIGDIHPGSGSTKYNEKFKSKATLTVDTSSSTAYMQLTRLSSEDSAVYYCARRDYWGQGTTLTVSS

SEQ ID NO: 34 - protein, 10 aa, Homo sapiens
GYILTTYWIT

SEQ ID NO: 35 - protein, 16 aa, Homo sapiens
DIHPGSGSTKYNEKFK

SEQ ID NO: 36 - protein, 3 aa, Homo sapiens
RDY

SEQ ID NO: 37 - protein, 107 aa, Homo sapiens
DIQMTQTTSSLSASLGDKVTISCRASQDISNYLSWYQQKPDGTVKLLIYWISRLHSGVPSRFSGSGSGTDFSLTISNLEQEDIATYFCQQGNSLPPTFGGGTKLEVK

SEQ ID NO: 38 - protein, 11 aa, Homo sapiens
RASQDISNYLS

SEQ ID NO: 39 - protein, 7 aa, Homo sapiens
WISRLHS

SEQ ID NO: 40 - protein, 9 aa, Homo sapiens
QQGNSLPPT

SEQ ID NO: 41 - protein, 119 aa, Homo sapiens
QVQLKQSGAELVKPGASVKISCKTSGYIFTDYYINWMKQRPGQGLEWIGKIGPGSGTTFYNEKFKGKATLTADKSSTTLYMQLSSLTAEDSAVYFCTRRGYYSSSYFYWGQGTTLTVSS

SEQ ID NO: 42 - protein, 10 aa, Homo sapiens
GYIFTDYYIN

SEQ ID NO: 43 - protein, 17 aa, Homo sapiens
KIGPGSGTTFYNEKFKG

SEQ ID NO: 44 - protein, 10 aa, Homo sapiens
RGYYSSSYFY

SEQ ID NO: 45 - protein, 113 aa, Homo sapiens
DIVMTQSPSSLSVSAGEKVIMSCKSSQSLLNSERQKNYLAWYQQKPGQSPKLLIYGASTRESGVPDRFTGSGSGTDFTLTISSVQAEDLAVYYCLNDHSYPLTFGAGTKLELK

SEQ ID NO: 46 - protein, 17 aa, Homo sapiens
KSSQSLLNSERQKNYLA

SEQ ID NO: 47 - protein, 7 aa, Homo sapiens
GASTRES

SEQ ID NO: 48 - protein, 9 aa, Homo sapiens
LNDHSYPLT

SEQ ID NO: 49 - protein, 118 aa, Homo sapiens
QVQLKQSGPGLVQPSQSLSITCTVSGFSLTSYGVHWVRQSPGKGLEWLGVIWSGGSTDYNAAFISRLSISKDNSKSQVFFKMNSLQADDTAIYYCARNGLLGNAMDYWGQGTSVTVSS

SEQ ID NO: 50 - protein, 10 aa, Homo sapiens
GFSLTSYGVH

SEQ ID NO: 51 - protein, 16 aa, Homo sapiens
VIWSGGSTDYNAAFIS

SEQ ID NO: 52 - protein, 10 aa, Homo sapiens
NGLLGNAMDY

SEQ ID NO: 53 - protein, 112 aa, Homo sapiens
DIVMSQSPSSLAVSAGEKVTMSCKSSQSLLSSRTRKNYLAWYQQKPGQSPKLLIYWASTRESGVPDRFTGSGSGTDFTLTISSVQTEDLAVYYCKQSYNLLTFGAGTTLELK

SEQ ID NO: 54 - protein, 17 aa, Homo sapiens
KSSQSLLSSRTRKNYLA

SEQ ID NO: 55 - protein, 7 aa, Homo sapiens
WASTRES

SEQ ID NO: 56 - protein, 8 aa, Homo sapiens
KQSYNLLT

SEQ ID NO: 57 - DNA, 354 bp, Homo sapiens
caggtgcagctgaggcagtcaggacctggcctagtgcagccctcacagagcctgtccatcacctgcacagtctctggtttctcattaactagttatggtgttcactgggttcgccagtctccaggaaagggtctggagtggctgggagtgatatggagtggtggaagcacagactataatgcagctttcatatccagactgagcatcagcaaggactattccaagagccaaattttccttacaatgaacagtctacaaactgatgacacagccatatattactgtgccagaaattcgagactgggaaactgctttgactactggggccaaggcaccactctcacagtctcctca

SEQ ID NO: 58 - DNA, 336 bp, Homo sapiens
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatcttcccgcgttcggaggggggaccaagctggaaataaaa

SEQ ID NO: 59 - DNA, 369 bp, Homo sapiens
caggttcagctgcagcagtctggacctgagctggtgaagcctggggcctcagtgaagatttcctgcaaggcttctggctacgtattcagtaactcctggatgaactgggtgaagcagaggcctggaaagggtcttgagtggattggacggattgatcctggagatggagatactaaatacaatgggaagttcaagggcaaggccacactgactgcagacaaatcctccagcacagcctacatgcaactcagcagcctgacatctgaggactctgcggtctacttctgtgtaagattttactacggcagtaactacgtagggtactactttgactattggggtcaaggcaccactctcacagtctcctca

SEQ ID NO: 60 - DNA, 336 bp, Homo sapiens
gatgttttgatgacccaaactccactctccctgcctgtcagtcttggagatcaagcctccatctcttgcagatctagtcagagcattgtacaaagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcccttgttcctccgacgttcggtggaggcaccaagctggaaatcaaa

SEQ ID NO: 61 - DNA, 372 bp, Homo sapiens
gaggtccagctgcaacaatctggacctgagctggtgaagcctggggcttcagtgaagatatcctgtaaggcttctggatacacgttcactgactactacatgagctggatgaagcagagccatggaaagagccttgagtggattggagatattaatcctaacaatggtgataatatctacaatcagaagttcaagggcaaggccacattgactgtagacaggtcctccagcacagcctacatggacctccgcagcctgacatctgaggactctgcagtctattactgtgcaagatggtattactacgttagtagcttcagctactggtacttcgatgtctggggcacagggaccacggtcaccgtctcctca

SEQ ID NO: 62 - DNA, 324 bp, Homo sapiens
gaaattgtgctcacccagtctccaaccaccatggctgcatctcccggggagaagatcactatcacctgcagtgccacctcaagtataaattccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtcgtactataccacgcacgttcggctcggggacaaagttggaaataaaa

SEQ ID NO: 63 - DNA, 345 bp, Homo sapiens
caggtccaactgaagcagtctggagctgagctggtgaagcctggggcttcagtgaagatatcctgcagggcttctggctacaccttcactgactactttataaactgggtgaagcagaggcctggacagggccttgagtggattggaaagattggtcctggaagtggtaatatttatcagaatgagaagtttaaggacaaggccacactgactacagacacatcctccagcacagcctacatgcagctcagcagtctaacatctgaggactctgcagtctatttctgtgcaagatggggaatacaacgagggtggggccaaggcaccactctcacagtctcctca

SEQ ID NO: 64 - DNA, 336 bp, Homo sapiens
gatgttgtgatgacccagactccactcactttgtcggttaccattggacaaccagcctccatctcttgcaagtcaagtcagagcctcttagatagtgatgggaagacatatttgaattggttgttacagaggccaggccagtctccaaagcgcctattctttctggtgtctaaacgggactctgaagtccctgacaggttcactggcagtggatctgggacagatttcacactgaaaatcagcagagtggaggctgaagatttgggagtttattattgctggcaagttacacattttccgtacacgttcggaggggggaccaagctggaaataaaa

SEQ ID NO: 65 - DNA, 336 bp, Homo sapiens
caggtccaactgcagcagcctggggctgaccttgtgaagcctggggcctcagtgaagatgtcctgtaaggcttctggctacatcctcaccacctattggataacctgggtgaagcagaggcctggacaaggccttgagtggattggagatattcatcctggtagtggtagtacaaagtacaatgagaaattcaagagtaaggccacactgactgtagacacatcctccagcacagcctacatgcagctcaccagactgtcatctgaggactctgcggtctattactgtgcaagaagggactactggggccaaggcaccactctcacagtctcctca

SEQ ID NO: 66 - DNA, 321 bp, Homo sapiens
gatatccagatgacacagactacatcctccctgtctgcctctctgggagacaaagtcaccatcagttgcagggcaagtcaggacattagcaattatttaagctggtatcagcagaaaccagatggaactgttaaactcctgatctactggatctcaagattacactcaggagtcccatcaaggttcagtggcagtgggtctggaacagatttttctctcaccattagcaacctggagcaagaagatattgccacttacttttgccaacagggtaattcgcttcctccgacgttcggtggaggcaccaagctggaagtcaaa

SEQ ID NO: 67 - DNA, 357 bp, Homo sapiens
caggtccagctgaagcagtctggagctgagctggtgaagcctggggcttcagtgaagatatcctgcaagacttctggctacatcttcactgactactacataaactggatgaaacaaaggcctggacagggccttgagtggattggaaagattggtcctggaagtggtactactttctacaatgagaagttcaagggcaaggccacactgactgcagacaaatcctccaccacactttatatgcagctcagcagcctgacagctgaggactctgcagtctatttctgtacaagaaggggttactacagtagtagctacttttactggggccaaggcaccactctcacagtctcctca

SEQ ID NO: 68 - DNA, 339 bp, Homo sapiens
gacatagtgatgacacagtctccatcctccctgagtgtgtcagcaggagagaaggtcataatgagctgcaagtccagtcagagtctgttaaacagtgaaagacaaaagaactacttggcctggtaccagcagaagccagggcagtctcctaaactgttgatctacggggcatccactagggaatcgggggtccctgatcgcttcacaggcagtggatctggaaccgatttcactcttaccatcagcagtgtgcaggctgaagacctggcagtttattactgtctgaatgatcatagttatccgctcacgttcggtgctgggaccaagctggaactgaaa

SEQ ID NO: 69 - DNA, 354 bp, Homo sapiens
caggtgcagctgaagcagtcaggacctggcctagtgcagccctcacagagcctgtccatcacctgcacagtctctggtttctcattgactagctatggtgtacactgggttcgccagtctccaggaaagggtctggagtggctgggagtcatatggagtggtggaagcacagactataatgcagctttcatatccagactgagcatcagcaaggacaattccaagagccaagttttctttaaaatgaacagtctgcaagctgatgacacagccatatattactgtgccagaaatggattacttgggaatgctatggactactggggtcaaggaacctcagtcaccgtctcctca

SEQ ID NO: 70 - DNA, 336 bp, Homo sapiens
gacattgtgatgtcacagtctccatcctccctggctgtgtcagcaggagagaaggtcactatgagctgcaaatccagtcagagtctgctcagcagtagaacccgaaaaaactacttggcttggtaccagcagaaaccagggcagtctcctaaactgctgatctactgggcatccactagggagtctggggtccctgatcgcttcacaggcagtggatctgggacagatttcactctcaccatcagcagtgtgcagactgaagacctggcagtttattactgcaagcaatcttataatctgctcacgttcggtgctgggaccacgctggagctgaaa

SEQ ID NO: 71 - DNA, 1050 bp, Homo sapiens
atgaactccttcaattataccacccctgattatgggcactatgatgacaaggataccctggacctcaacacccctgtggataaaacttctaacacgctgcgtgttccagacatcctggccttggtcatctttgcagtcgtcttcctggtgggagtgctgggcaatgccctggtggtctgggtgacggcattcgaggccaagcggaccatcaatgccatctggttcctcaacttggcggtagccgacttcctctcctgcctggcgctgcccatcttgttcacgtccattgtacagcatcaccactggccctttggcggggccgcctgcagcatcctgccctccctcatcctgctcaacatgtacgccagcatcctgctcctggccaccatcagcgccgaccgctttctgctggtgtttaaacccatctggtgccagaacttccgaggggccggcttggcctggatcgcctgtgccgtggcttggggtttagccctgctgctgaccataccctccttcctgtaccgggtggtccgggaggagtactttccaccaaaggtgttgtgtggcgtggactacagccacgacaaacggcgggagcgagccgtggccatcgtccggctggtcctgggcttcctgtggcctctactcacgctcacgatttgttacactttcatcctgctccggacgtggagccgcagggccacgcggtccaccaagacactcaaggtggtggtggcagtggtggccagtttctttatcttctggttgccctaccaggtgacggggataatgatgtccttcctggagccatcgtcacccaccttcctgctgctgaataagctggactccctgtgtgtctcctttgcctacatcaactgctgcatcaaccccatcatctacgtggtggccggccagggcttccagggccgactgcggaaatccctccccagcctcctccggaacgtgttgactgaagagtccgtggttagggagagcaagtcattcacgcgctccacagtggacactatggcccagaagacccaggcagtg

SEQ ID NO: 72 - protein, 350 aa, Homo sapiens
MNSFNYTTPDYGHYDDKDTLDLNTPVDKTSNTLRVPDILALVIFAVVFLVGVLGNALVVWVTAFEAKRTINAIWFLNLAVADFLSCLALPILFTSIVQHHHWPFGGAACSILPSLILLNMYASILLLATISADRFLLVFKPIWCQNFRGAGLAWIACAVAWGLALLLTIPSFLYRVVREEYFPPKVLCGVDYSHDKRRERAVAIVRLVLGFLWPLLTLTICYTFILLRTWSRRATRSTKTLKVVVAVVASFFIFWLPYQVTGIMMSFLEPSSPTFLLLNKLDSLCVSFAYINCCINPIIYVVAGQGFQGRLRKSLPSLLRNVLTEESVVRESKSFTRSTVDTMAQKTQAV